(19) United States
(12) Patent Application Publication
(10) Pub. No.: US 2012/0156294 A1
(43) Pub. Date: Jun. 21, 2012

(54) Pharmaceutical Compositions of Selective Factor Xa Inhibitors for Oral Administration

(76) Inventors: Thomas W. Leonard, Wilmington, NC (US); David C. Coughlan, Kildare (IE); Alan Cullen, Dublin (IE)

(21) Appl. No.: 13/242,601

(22) Filed: Sep. 23, 2011

Related U.S. Application Data
(60) Provisional application No. 61/423,261, filed on Dec. 15, 2010.

(51) Int. Cl. A61K 9/24; A61P 7/02; A61K 31/702

(52) U.S. Cl. 424/472; 514/54

(57) ABSTRACT

The present invention provides pharmaceutical compositions for oral administration comprising a therapeutically effective amount of a selective factor Xa inhibitor or a pharmaceutically acceptable salt thereof and an enhancer, wherein the enhancer is a medium chain fatty acid or a salt, ester, ether, or derivative of a medium chain fatty acid and has a carbon chain length of from 4 to 20 carbon atoms. The present invention also provides a method for obtaining a reproducible bioavailability of selective factor Xa inhibitor in a subject after oral administration comprising orally administering a pharmaceutical composition as described above.

This application claims the benefit of U.S. Provisional Application Ser. No. 61/423,261 filed Dec. 15, 2010, the disclosure of which is incorporated by reference herein in its entirety.

### FIELD OF THE INVENTION

The present invention generally relates to orally administered pharmaceutical compositions of selective factor Xa inhibitors, e.g., oligosaccharides such as fondaparinux.

### BACKGROUND OF THE INVENTION

Today, anticoagulants are widely used to prevent and treat a variety of thromboembolic events. Currently, available anticoagulants for treatment include unfractionated heparin (UFH), low molecular weight heparin (LMWH), and vitamin K antagonists (VKAs) (e.g., warfarin). More recently, synthetic pentasaccharides (e.g., fondaparinux (Arixtra®)) have also been developed as effective anticoagulants. The advantage of fondaparinux over LMWH or UFH is that the risk for heparin-induced thrombocytopenia is substantially reduced as fondaparinux inhibits factor Xa via binding to antithrombin III and does not inhibit thrombin or possess other activities of heparin.

Although anticoagulants are effective in treating and reducing the risk of thromboembolic disease, they are associated with significant drawbacks that limit their use and acceptability in the clinical setting. The traditional anticoagulants are administered parenterally and require frequent monitoring and subsequent dose adjustment. For example, UFH, LMWH, and fondaparinux are administered parenterally, which is inconvenient and expensive for long-term use, particularly outside of the hospital setting where visits to or from a health care professional may be required. Vitamin K antagonists are available for oral administration. However, they usually have a narrow therapeutic window and unpredictable pharmacology, and require close monitoring and dose adjustment to ensure that anticoagulant effects remain within the therapeutic range.

Different approaches have been proposed to develop orally administered anticoagulants. One approach is to develop small molecule direct factor Xa inhibitors. Unlike the more traditional anticoagulants (UFH, LMWH, and VKAs) that target multiple enzymes in the coagulation cascade, the new drugs inhibit single enzymes. Rivaroxaban, a direct factor Xa inhibitor, was recently approved for oral administration. Other oral factor Xa inhibitors in clinical development include apixaban (Bristol-Myers Squibb), and 813893 (GlaxoSmithKline).

Despite the recent promising development of direct factor Xa inhibitors, there is a continuing need for development of novel pharmaceutical formulations of selective factor Xa inhibitors suitable for oral administration, which not only offer the convenience of oral dosing, but also provide a reproducible and predictable bioavailability of the active ingredient.

### SUMMARY OF THE INVENTION

The present invention provides a pharmaceutical composition for oral administration comprising a therapeutically effective amount of a selective factor Xa inhibitor or a pharmaceutically acceptable salt thereof and an enhancer, wherein the enhancer is a medium chain fatty acid or a salt, ester, ether, or derivative of a medium chain fatty acid and has a carbon chain length of from 4 to 20 carbon atoms. In some embodiments, the selective factor Xa inhibitor is fondaparinux or a pharmaceutically acceptable salt thereof.

Another aspect of the present invention provides a method of treating or preventing a medical condition, comprising administering to a subject in need of treatment or prevention a therapeutically effective amount of a selective factor Xa inhibitor or a pharmaceutically acceptable salt thereof together with an enhancer, wherein the enhancer is a medium chain fatty acid or a salt, ester, ether, or derivative of a medium chain fatty acid and has a carbon chain length of from 4 to 20 carbon atoms.

Another aspect of the present invention provides a process for manufacturing a solid oral dosage form of a pharmaceutical composition comprising the steps of: a) blending a selective factor Xa inhibitor or a pharmaceutically acceptable salt thereof with an enhancer, and optionally additional excipients, to form a blend; wherein the enhancer is a medium chain fatty acid or a salt, ester, ether, or derivative of a medium chain fatty acid and has a carbon chain length of from 4 to 20 carbon atoms; and b) forming a solid oral dosage from the blend by i) directly compressing the blend to form the solid oral dosage form, or ii) granulating the blend to form a granulate for incorporation into the solid oral dosage form, or iii) spray drying the blend to form a multiparticulate for incorporation into the solid oral dosage form.

Another aspect of the present invention provides a pharmaceutical composition of a selective factor Xa inhibitor or a pharmaceutically acceptable salt thereof for oral administration in the form of a stable, transparent drug delivery composition, wherein the drug delivery composition comprises (a) from about 1 to about 80 weight percent of a pharmaceutically acceptable oil; (b) from about 3 to about 98 weight percent (e.g., from about 3 to about 96.5 weight percent) surface active agents; (c) from about 2 to about 60 weight percent polyethylene glycol; and (d) from about 0.5 to about 15 weight percent water; wherein the ratio of the polyethylene glycol to water is at least 2:1.

One aspect of the present invention describes a pharmaceutical composition of a selective factor Xa inhibitor or a pharmaceutically acceptable salt thereof for oral administration which is an emulsion composition, wherein an internal phase of the emulsion composition contains a therapeutically effective amount of a selective factor Xa inhibitor or a pharmaceutically acceptable salt thereof; and the internal phase comprises a polar, nonaqueous, oxygen-containing, pharmaceutically acceptable liquid selected from the group consisting of C₂-C₃₀ polyhydric alcohols, poly(ethylene or propylene) glycols with 4-200 repeating units, C₂-C₃₀ ester derivatives thereof, and C₁-C₅ ether derivatives thereof.

Another aspect of the present invention provides a pharmaceutical composition of a selective factor Xa inhibitor for oral administration which is a water-in-oil microemulsion composition, wherein the microemulsion composition converts to an oil-in-water emulsion by the addition of water and the microemulsion composition comprises (a) up to about 20 volume percent of an internal dispersed aqueous phase containing a therapeutically effective amount of a selective factor Xa inhibitor or a pharmaceutically acceptable salt thereof, (b) from about 30 to about 99 volume percent of a continuous oil phase comprising mono and di-esters of propylene glycol having from 15 to 40 carbon atoms, and (c) from about 1 to about 70 volume percent of a surfactant or mixture of surfactants, wherein the surfactant or surfactant mixture has a hydrophilic-lipophilic balance (HLB) value of from 7 to 14.

One aspect of the present invention describes a pharmaceutical composition of a selective factor Xa inhibitor for oral administration which is a water-in-oil microemulsion composition, wherein the microemulsion composition converts to an oil-in-water emulsion by the addition of water and the microemulsion composition comprises (a) up to about 60 volume percent, based upon the total volume of the microemulsion, of an internally dispersed aqueous phase containing a therapeutically effective amount of a selective factor Xa inhibitor or a pharmaceutically acceptable salt thereof; (b) from about 5 to about 90 volume percent of a continuous oil phase comprising at least one pharmaceutically acceptable oil; and (c) from 1 to about 70 volume percent of a surfactant or mixture of surfactants, wherein the surfactant or surfactant mixture has a HLB value of from 7 to 14.

Another aspect of the present invention provides a pharmaceutical composition of a selective factor Xa inhibitor for oral administration which is a water-in-oil microemulsion composition, wherein the microemulsion composition comprises (a) from about 5 to about 99 volume percent of an oil phase comprising at least one pharmaceutically acceptable oil; (b) up to about 60 volume percent of an aqueous phase comprising water; (c) a therapeutically effective amount of a selective factor Xa inhibitor or a pharmaceutically acceptable salt thereof; (d) from about 1 to about 70 volume percent of a mixture of surfactants having a combined HLB value of from about 7 to about 14 comprising (i) a low HLB surfactant having a HLB below 8, said low HLB surfactant being at least 40 percent by weight of a C₉ monoglyceride, C₁₀ monoglyceride, C₁₁ monoglyceride, C₁₂ monoglyceride, or C₁₃ monoglyceride, and (ii) at least one surfactant having a HLB value above about 8.

One aspect of the present invention provides a pharmaceutical composition of a selective factor Xa inhibitor for oral administration which is a water-in-oil microemulsion composition, wherein the microemulsion composition comprises (a) up to about 60 volume percent of an internal dispersed aqueous phase containing a therapeutically effective amount of a selective factor Xa inhibitor or a pharmaceutically acceptable salt thereof; (b) from about 5 to about 99 volume percent of a continuous oil phase comprising at least one pharmaceutically acceptable oil comprising a C₉₋₈₃ triglyceride, a C₇₋₅₅ mono- and di-ester of propylene glycol, or mixtures thereof; and (c) from about 1 to about 70 volume percent of a surfactant or surfactant mixture comprising a C₈ fatty acid salt, wherein the surfactant or surfactant mixture has a HLB value of at least 7.

Another aspect of the present invention provides a method for obtaining a reproducible bioavailability of a selective factor Xa inhibitor in a subject after oral administration, comprising orally administering a pharmaceutical composition of the invention to said subject.

A further aspect of the present invention provides a pharmaceutical composition, which is effective in providing therapeutically effective blood levels of a selective factor Xa inhibitor or a pharmaceutically acceptable salt thereof to a subject when administered to a gastrointestinal tract, comprising:

(i) a therapeutically effective amount of a selective factor Xa inhibitor or a pharmaceutically acceptable salt thereof; and

(ii) at least one enhancer;

wherein the pharmaceutical composition provides rapid release of the selective factor Xa inhibitor or a pharmaceutically acceptable salt thereof and the enhancer after the pharmaceutical composition enters the intestine of a subject; and
wherein the pharmaceutical composition, in the form of a dosage form with coating, provides an in vitro dissolution of at least 80% of the selective factor Xa inhibitor and the enhancer in 40 minutes and/or at least 90% of the selective factor Xa inhibitor and the enhancer in 45 minutes.

A further aspect of the present invention provides a pharmaceutical composition, which is effective in providing therapeutically effective blood levels of a selective factor Xa inhibitor or a pharmaceutically acceptable salt thereof to a subject when administered to a gastrointestinal tract, comprising:

(i) a therapeutically effective amount of a selective factor Xa inhibitor or a pharmaceutically acceptable salt thereof; and

(ii) at least one enhancer;

wherein the pharmaceutical composition provides a substantially similar release rate of the selective factor Xa inhibitor or a pharmaceutically acceptable salt thereof and the enhancer after the pharmaceutical composition enters the intestine of a subject; and
wherein the substantially similar release rate is a ratio of the time for a percentage of the therapeutically active agent to be released in an in vitro dissolution from a dosage form of the pharmaceutical composition with coating to the time for the same percentage of the enhancer to be released of about 1.3 to about 0.7.

### BRIEF DESCRIPTION OF THE DRAWINGS

FIG. 1 shows the plasma concentration levels of different GIPET™ (“Gastrointestinal Permeation Enhancement Technology”) formulations of fondaparinux in dogs following intraduodenal administration.

FIG. 2 shows the bioavailability of different GIPET™ formulations of fondaparinux in dogs following intraduodenal administration.

FIG. 3 shows the fondaparinux mean plasma concentration profiles (0-24 hours) after administration in male beagle dogs.

FIG. 4 shows the mean bioavailability (% Fᵣₑₗ) of fondaparinux after administration of GIPET® I tablet formulations in beagle dogs relative to subcutaneous administration.

FIG. 5 shows the bioavailability (% Fᵣₑₗ) of fondaparinux after administration of GIPET® I tablet formulations in beagle dogs relative to subcutaneous administration.

FIG. 6 shows phase 1 fondaparinux plasma concentration profiles (0-24 hrs) for individual dogs.

FIG. 7 shows phase 2 fondaparinux plasma concentration profiles (0-24 hrs) for individual dogs.

FIG. 8 shows phase 3 fondaparinux plasma concentration profiles (0-24 hrs) for individual dogs.

FIG. 9 shows the mean fondaparinux plasma concentration profiles (24 hours) of Test Items after administration in Dog M1 (5603251).

FIG. 10 shows the mean fondaparinux plasma concentration profiles (24 hours) of Test Items after administration in Dog M2 (5723906).

FIG. 11 shows the mean fondaparinux plasma concentration profiles (24 hours) of Test Items after administration in Dog M3 (1419995).

FIG. 12 shows the mean fondaparinux plasma concentration profiles (24 hours) of Test Items after administration in Dog M4 (1420004).

FIG. 13 shows the mean fondaparinux plasma concentration profiles (24 hours) of Test Items after administration in Dog M5 (1423135).

FIG. 14 shows the mean fondaparinux plasma concentration profiles (24 hours) of Test Items after administration in Dog M6 (1424930).

FIG. 15 shows the normalized dissolution profile for the 12.5 mg fondaparinux tablet.

FIG. 16 shows the normalized dissolution profile for the 20 mg fondaparinux tablet.

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

All patents, patent applications and publications referred to herein are incorporated by reference in their entirety. In case of a conflict in terminology, the present specification is controlling.

The terminology used in the description of the invention herein is for the purpose of describing particular embodiments only and is not intended to be limiting of the invention.

As used in the description of the embodiments of the invention and the appended claims, the singular forms “a”, “an” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “consists essentially of” (and grammatical variants), as applied to the compositions of this invention, means the composition can contain additional components as long as the additional components do not materially alter the composition. The term “materially altered,” as applied to a composition, refers to an increase or decrease in the therapeutic effectiveness of the composition of at least about 20% or more as compared to the effectiveness of a composition consisting of the recited components.

Unless the context indicates otherwise, it is specifically intended that the various features of the invention described herein can be used in any combination. For example, features described in relation to one embodiment may also be applicable to and combinable with other embodiments and aspects of the invention.

Moreover, the present invention also contemplates that in some embodiments of the invention, any feature or combination of features set forth herein can be excluded or omitted.

The term “tablet” as used herein includes, but is not limited to, immediate release (IR) tablets, sustained release (SR) tablets, matrix tablets, multilayer tablets, multilayer matrix tablets, extended release tablets, delayed release tablets and pulsed release tablets any or all of which may optionally be coated with one or more coating materials, including polymer coating materials, such as enteric coatings, rate-controlling coatings, semi-permeable coatings and the like. The term “tablet” also includes osmotic delivery systems in which a drug compound is combined with an osmagent (and optionally other excipients) and coated with a semi-permeable membrane, the semi-permeable membrane defining an orifice through which the drug compound may be released. Tablet solid oral dosage forms that may be useful in the practice of the invention include those selected from the group consisting of IR tablets, SR tablets, coated IR tablets, coated SR tablets, matrix tablets, coated matrix tablets, multilayer tablets, coated multilayer tablets, multilayer matrix tablets and coated multilayer matrix tablets. In some embodiments, a tablet dosage form is an enteric-coated tablet dosage form. In some embodiments, a tablet dosage form is an enteric-coated rapid onset tablet dosage form.

Capsule solid oral dosage forms that may be useful in the practice of the present invention include those selected from the group consisting of IR capsules, SR capsules, coated IR capsules, and coated SR capsules including delayed release capsules. Capsules may be filled with powders, granules, multiparticulates, tablets, semi-solids, or liquids. In some embodiments, a capsule dosage form is an enteric-coated capsule dosage form. In some embodiments, a capsule dosage form is an enteric-coated rapid onset capsule dosage form. Capsules may be made of hard gelatin, soft gelatin, starch, cellulose polymers, or other materials as known to the art.

The term “multiparticulate” as used herein means a plurality of discrete particles, pellets, mini-tablets and mixtures or combinations thereof. If the oral form is a multiparticulate capsule, hard or soft gelatin capsules or capsules of other materials can suitably be used to contain the multiparticulate. In some embodiments, a sachet can suitably be used to contain the multiparticulate. In some embodiments, the multiparticulate may be coated with a layer containing rate controlling polymer material. In some embodiments, a multiparticulate oral dosage form according to the invention may comprise a blend of two or more populations of particles, pellets, or mini-tablets having different in vitro and/or in vivo release characteristics. For example, a multiparticulate oral dosage form may comprise a blend of an instant release component and a delayed release component contained in a suitable capsule.

In some embodiments, the multiparticulate and one or more auxiliary excipient materials can be compressed into tablet form such as a multilayer tablet. In some embodiments, a multilayer tablet may comprise two layers containing the same or different levels of the same active ingredient having the same or different release characteristics. In some embodiments, a multilayer tablet may contain different active ingredient in each layer. Such a tablet, either single layered or multilayered, can optionally be coated with a controlled release polymer so as to provide additional controlled release properties. In some embodiments, multiparticulate dosage form comprises a capsule containing delayed release rapid onset minitablets. In some embodiments, a multiparticulate dosage form comprises a delayed release capsule comprising instant release minitablets. In some embodiments, a multiparticulate dosage form comprises a capsule comprising delayed release granules. In some embodiments, a multiparticulate dosage form comprises a delayed release capsule comprising instant release granules.

The term “emulsion” as used herein means a suspension or dispersion of one liquid within a second immiscible liquid. In some embodiments, the emulsion is an oil-in-water or water-in-oil-in-water emulsion.

The term, “microemulsion” as used herein means a solution in which the hydrophobic (oil-like) phase and the hydrophilic (water-like) phase and a surfactant form micelle structures. Such dispersions are clear and stable over time. In certain embodiments, the micelles have an average diameter of about 1 micron or less.

In addition, “emulsion” or “microemulsion” as used herein includes a hydrophilic or a hydrophobic liquid which, on dilution with a hydrophobic or a hydrophilic liquid respectively, forms an emulsion or a microemulsion. In some embodiments, “emulsion” or “microemulsion” as used herein may include solid or semi-solid materials which may be liquid at higher temperatures. For example, the material may be solid at room temperature. At about body temperature (about 37° C.), the material may be liquid.

By the terms “treat,” “treating,” or “treatment of” (and grammatical variations thereof) it is meant that the severity of the subject's condition is reduced, at least partially improved, or stabilized and/or that some alleviation, mitigation, decrease, or stabilization in at least one clinical symptom and/or parameter is achieved and/or there is a delay in the progression of the disease or disorder.

The terms “prevent,” “preventing,” and “prevention” (and grammatical variations thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective amount,” as used herein, refers to an amount that imparts a desired effect, which is optionally a therapeutic or prophylactic effect.

A “treatment effective” amount, as used herein, is an amount that is sufficient to provide some improvement or benefit to the subject. Alternatively stated, a “treatment effective” amount is an amount that will provide some alleviation, mitigation, decrease, or stabilization in at least one clinical symptom in the subject. Those skilled in the art will appreciate that the therapeutic effects need not be complete or curative, as long as some benefit is provided to the subject.

A “prevention effective” amount, as used herein, is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, a “therapeutically effective” or “therapeutically acceptable” amount refers to an amount that will elicit a therapeutically useful response in a subject. The therapeutically useful response may provide some alleviation, mitigation, or decrease in at least one clinical symptom in the subject. The terms also include an amount that will prevent or delay at least one clinical symptom in the subject and/or reduce and/or delay the severity of the onset of a clinical symptom in a subject relative to what would occur in the absence of the methods of the invention. Those skilled in the art will appreciate that the therapeutically useful response need not be complete or curative or prevent permanently, as long as some benefit is provided to the subject.

“Subjects” according to the present invention include mammals, avians, reptiles, amphibians, and fish. Mammalian subjects include but are not limited to humans, non-human mammals, non-human primates (e.g., monkeys, chimpanzees, baboons, etc.), dogs, cats, mice, hamsters, rats, horses, cows, pigs, rabbits, sheep and goats. In particular embodiments, the subject is a laboratory animal. Human subjects include neonates, infants, juveniles, adults, and geriatric subjects. In certain embodiments, the subject is in need of the methods of the present invention, e.g., has a thromboembolic disorder. In other embodiments, the subject has, may have, or is at risk for a thromboembolic disorder.

“Stable,” as used herein with respect to pharmaceutical compositions, refers to a composition that degrades no more than 10% when stored for one month at −20° C., e.g., at 4° C., e.g., at room temperature, and a relative humidity of 20% to 80%.

“Reproducible,” as used herein, refers to pharmacokinetic characteristics of the pharmaceutical compositions of the invention that are consistent from subject to subject. A reproducible pharmacokinetic characteristic, e.g., bioavailability, Cₘₐₓ, or AUC, is one that has a coefficient of variation of less than about 60%, e.g., less than about 60, 55, 50, 45, 40, 35, 30, 25, 20, or 15% or less.

As used herein, a “derivative of a medium chain fatty acid” refers to a fatty acid derivative having at least one carbon chain of from 4 to 20 carbon atoms in length. This carbon chain may be characterized by various degrees of saturation. In other words, the carbon chain may be, for example, fully saturated or partially unsaturated (i.e., containing one or more carbon-carbon multiple bonds). The term “fatty acid derivative” is meant to encompass acyl derivatives such as esters, acid halides, anhydrides, amides and nitrites, and also ethers and glycerides such as mono-, di- or tri-glycerides. The term “fatty acid derivative” is meant to further encompass medium chain fatty acids wherein the end of the carbon chain opposite the acid group (or derivative) is also functionalized with one of the above mentioned moieties (i.e., ester, acid halide, anhydride, amide, nitrile, ether and glyceride moieties). Such difunctional fatty acid derivatives thus include for example diacids and diesters (the functional moieties being of the same kind) and also difunctional compounds comprising different functional moieties, such as amino acids and amino acid derivatives (for example a medium chain fatty acid, or an ester or a salt thereof, comprising an amide moiety at the opposite end of the fatty acid carbon chain to the acid (or ester or salt thereof).

The present invention provides a pharmaceutical composition for oral administration comprising, consisting essentially of, or consisting of a therapeutically effective amount of a selective factor Xa inhibitor or a pharmaceutically acceptable salt thereof and an enhancer, wherein the enhancer is a medium chain fatty acid or a salt, ester, ether, or derivative of a medium chain fatty acid and has a carbon chain length of from 4 to 20 carbon atoms. In certain embodiments, the medium chain fatty acid enhancer is the only enhancer present in the composition.

The present invention provides a solid oral dosage comprising, consisting essentially of, or consisting of the pharmaceutical composition of the invention. In certain embodiments, the solid oral dosage form is a tablet, a multiparticulate, or a capsule.

In some embodiments, the selective factor Xa inhibitor is an oligosaccharide. In another embodiment, the selective factor Xa inhibitor is a pentasaccharide. In some embodiments, the selective factor Xa inhibitor is fondaparinux or a pharmaceutically acceptable salt thereof.

As used herein, “selective factor Xa inhibitor” refers to a compound which selectively inhibits factor Xa directly or indirectly (e.g., via antithrombin III) but does not possess a significant activity towards thrombin. The term “does not possess a significant activity” refers to a compound that reduces thrombin activity by less than 20%, e.g., less than 15%, 10%, or 5%. In some embodiments, the selective factor Xa inhibitor possesses no inhibitory activity towards thrombin. In some embodiments, the selective factor Xa inhibitor is an indirect inhibitor. In some embodiments, the selective factor Xa inhibitor is one that exhibits poor oral bioavailability, e.g., less than 5%, 4%, 3%, 2%, or 1% oral bioavailability. In some embodiments, the selective factor Xa inhibitor is an oligosaccharide, e.g., a pentasaccharide. Exemplary synthetic selective factor Xa inhibitors include, but are not limited to, fondaparinux and pharmaceutically acceptable salts thereof (the structure of fondaparinux is shown below), antistasin, tick anticoagulant peptide, yagin, apixaban, otamixaban, rivaroxaban, NAP-5, TAP, rNAPc-2, TFPI, DX-9065a, YM-60828, RPR-120844, BX-807834, and compounds described in EP 84999, EP 529715, EP 621282, U.S. Pat. Nos. 6,541,488, 6,391,339, 6,369,080, 6,262,047, and 6,133,256, and U.S. Published Application No. 2006/0122151, which are incorporated by reference in their entireties.

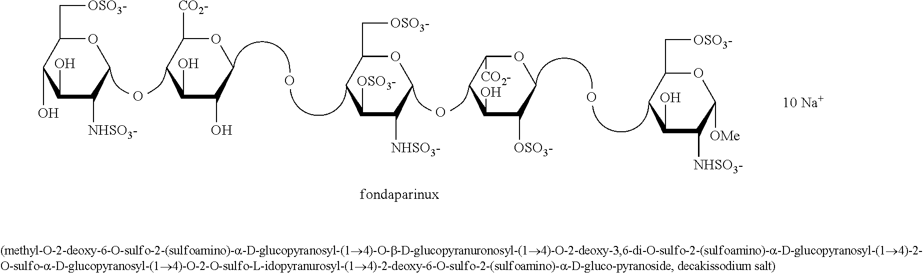

A salt of a selective factor Xa inhibitor may be prepared by combining the compound in its free acid or base form with a suitable organic or inorganic acid or base and isolating the salt thus formed. For example, when the selective factor Xa inhibitor is fondaparinux (in the sodium salt form), the salt is formed by reacting the free acid form of fondaparinux with a suitable inorganic or organic base. The term “pharmaceutically acceptable salt” refers to a relatively non-toxic, inorganic or organic acid or base addition salt of a compound of the present invention (see, e.g., Berge et al., J. Pharm. Sci. 66:1-19, 1977).

Representative salts of the compounds of the present invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, undecanoate, and the like.

Base salts include, for example, alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups in the conjugate base may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides; aralkyl halides like benzyl and phenethyl bromides, and the like.

In some embodiments, the enhancer is a salt of a medium chain fatty acid which has a carbon chain length of from 6 to 20 carbon atoms. In some embodiments, the enhancer (e.g., the medium chain fatty acid or the salt of a medium chain fatty acid) is solid at room temperature. In some embodiments, the chain length is from 8 to 14 carbon atoms. In other embodiments, the enhancer is a sodium salt of a medium chain fatty acid. In some embodiments, the enhancer is selected from the group consisting of sodium caprylate, sodium caprate and sodium laurate. Exemplary enhancers are further described in U.S. Pat. Nos. 7,658,938 and 7,670,626 and U.S. Published Application Nos. 2003/0091623 and 2007/0238707, which are incorporated by reference in their entirety.

In some embodiments, the pharmaceutical composition of the invention comprises, consists essentially of, or consists of a therapeutically effective amount of a selective factor Xa inhibitor or a pharmaceutically acceptable salt thereof, an enhancer, wherein the enhancer is a medium chain fatty acid or a salt, ester, ether, or derivative of a medium chain fatty acid and has a carbon chain length of from 8 to 14 carbon atoms, and one or more auxiliary excipients. In other embodiments, the pharmaceutical composition of the invention comprises, consists essentially of, or consists of a therapeutically effective amount of a selective factor Xa inhibitor or a pharmaceutically acceptable salt thereof, an enhancer, wherein the enhancer is a medium chain fatty acid or a salt thereof and has a carbon chain length of from 8 to 14 carbon atoms, and one or more auxiliary excipients. In other embodiments, the pharmaceutical composition of the invention comprises, consists essentially of, or consists of a therapeutically effective amount of a selective factor Xa inhibitor or a pharmaceutically acceptable salt thereof, an enhancer, wherein the enhancer is a medium chain fatty acid salt and has a carbon chain length of from 8 to 14 carbon atoms, and one or more auxiliary excipients. In each of these embodiments, the factor Xa inhibitor can be fondaparinux and/or the enhancer can be sodium caprate.

In some embodiments, the enhancer is present in a ratio of from 1:100,000 to 10:1 (inhibitor:enhancer), e.g., from 1:1000 to 5:1, e.g., from 1:300 to 1:1. In some embodiments, the dosage form is a tablet, a capsule, or a multiparticulate dosage form. In some embodiments, the dosage form is a controlled release dosage form. In some embodiments, the tablet further comprises a rate controlling polymer material. In some embodiments, the rate-controlling polymer is hydroxypropyl methylcellulose (HPMC). In some embodiments, the rate-controlling polymer is a polymer of acrylic or methacrylic acid or their respective esters or copolymers of acrylic or methacrylic acid and/or their respective esters.

In some embodiments, the selective factor Xa inhibitor and enhancer and at least one auxiliary excipient are compressed into tablet form prior to coating with a rate controlling polymer. In some embodiments, the selective factor Xa inhibitor and enhancer and at least one auxiliary excipient are compressed into tablet form prior to coating with a delayed release polymer. In some embodiments, the selective factor Xa inhibitor, the enhancer, the rate controlling polymer and at least one auxiliary excipient are compressed to form a controlled release matrix tablet. In some embodiments, the controlled release matrix tablet is coated with a rate-controlling polymer. In some embodiments, the controlled release matrix is coated with a delayed release polymer. In some embodiments, the selective factor Xa inhibitor, the enhancer and at least one auxiliary excipient are compressed into the form of a multilayer tablet prior to coating with a rate controlling-polymer. In some embodiments, the selective factor Xa inhibitor, the enhancer and at least one auxiliary excipient are compressed into the form of a multilayer tablet prior to coating with a delayed release polymer. Yet, in another embodiment, the selective factor Xa inhibitor and enhancer are dispersed in the rate-controlling polymer material and compressed into the form of a multilayer tablet. In some embodiments, the multilayer tablet is coated with a rate-controlling polymer. In some embodiments, the multilayer tablet is coated with a delayed release polymer.

In some embodiments, the selective factor Xa inhibitor, the enhancer, at least one auxiliary excipient, and the rate-controlling polymer material are combined into a multiparticulate form. In some embodiments, the multiparticulate form comprises discrete particles, pellets, minitablets, or combinations thereof. In some embodiments, the pharmaceutical composition of the present invention comprises a blend of two or more populations of particles, pellets or mini-tablets having different in vitro or in vivo release characteristics. In some embodiments, the multiparticulate is encapsulated in hard or soft gelatin capsules. In another embodiment, the capsule is coated with a rate-controlling polymer. In some embodiments, the capsule is coated with a delayed release polymer. In some embodiments, the multiparticulate is incorporated into a sachet.

In some embodiments, the discrete particles or pellets are compressed into tablet form. In some embodiments, the tablet form is coated with a rate controlling polymer material. Yet, in another embodiment, the tablet form is coated with a delayed release polymer. In some embodiments, the discrete particles or pellets are compressed into a multilayer tablet. In some embodiments, the multilayer tablet is coated with a rate controlling material. In some embodiments, the multilayer tablet is coated with a delayed release polymer.

In the case of any of the above-mentioned embodiments, a controlled release coating (e.g., an enteric coating) may be applied to the final dosage form (capsule, tablet, multilayer tablet etc.). The controlled release coating may typically comprise a rate controlling polymer material as defined above. The dissolution characteristics of such a coating material may be pH dependent or independent of pH.

The pharmaceutical compositions of the invention can comprise one or more auxiliary excipients, such as for example rate-controlling polymeric materials, diluents, lubricants, disintegrants, plasticizers, anti-tack agents, opacifying agents, glidants, pigments, flavorings, and such like. As will be appreciated by those skilled in the art, the exact choice of excipients and their relative amounts will depend to some extent on the final dosage form.

One excipient that can be included in the composition is one or more saccharides. Any suitable saccharide may be used in the composition of the present invention. As used herein, the “saccharides” used in the invention include sugar alcohols, monosaccharides, disaccharides, and oligosaccharides. Exemplary sugar alcohols include, but not limited to, xylitol, mannitol, sorbitol, erythritol, lactitol, pentitol, and hexitol. Exemplary monosaccharides include, but are not limited to, glucose, fructose, aldose and ketose. Exemplary disaccharides include, but are not limited to, sucrose, isomalt, lactose, trehalose, and maltose. Exemplary oligosaccharides include, but are not limited to, fructo-oligosaccharides, inulin, galacto-ologosaccharides, and mannan-oligosaccharides. In some embodiments, the saccharide is sorbitol, mannitol, or xylitol. In some embodiments, the saccharide is sorbitol. In some embodiments, the saccharide is sucrose.

Any suitable amounts of saccharide may be added in the compositions of the present invention. In some embodiments of the present invention, the ratio of the enhancer and saccharide may be adjusted to achieve a desired dissolution rate and/or compressibility of the resulting pharmaceutical composition. In some embodiments, the ratio of the enhancer and saccharide is 2:1 to 20:1. According to some embodiments, the ratio of the enhancer and saccharide is about 4:1 to 6:1. In another embodiment, the ratio of the enhancer and saccharide is about 5:1.

Any suitable grade of saccharide may be used in the composition of the present invention. However, in some embodiments, the selection of the grade of saccharide may be dependent upon the particle size distribution (PSD) of a specific grade of saccharide. Further, in another embodiment, the specific grade of the saccharide may affect the characteristics of the resulting pharmaceutical composition such as dissolution rate and/or compressibility. In some embodiments, the selection of the grade of saccharide is dependent upon the PSD of other excipients and the therapeutically active ingredient. In some embodiments, the saccharide is Parteck SI 150 (Merck KGaA, Darmstadt, Germany), a directly compressible sorbitol. In other embodiments, the saccharide is Parteck SI 400 (Merck KGaA, Darmstadt, Germany).

Suitable diluents include, for example, pharmaceutically acceptable inert fillers such as microcrystalline cellulose, lactose, dibasic calcium phosphate, saccharides, and/or mixtures of any of the foregoing. Examples of diluents include microcrystalline cellulose such as that sold under the Trademark Avicel (FMC Corp., Philadelphia, Pa.), for example, Avicel™ pH101, Avicel™ pH102 and Avicel™ pH112; lactose such as lactose monohydrate, lactose anhydrous and Pharmatose DCL21; dibasic calcium phosphate such as Emcompress; mannitol; starch; sorbitol; sucrose; glucose; and combinations and mixtures thereof.

Suitable lubricants, including agents that act on the flowability of the powder to be compressed are, for example, colloidal silicon dioxide such as Aerosil™ 200; talc; stearic acid; magnesium stearate; calcium stearate; and combinations and mixtures thereof.

Suitable disintegrants include, for example, lightly crosslinked polyvinyl pyrrolidone, corn starch, potato starch, maize starch and modified starches, croscarmellose sodium, crospovidone, sodium starch glycolate, and combinations and mixtures thereof.

The term “rate controlling polymer material” as used herein includes hydrophilic polymers, hydrophobic polymers and mixtures of hydrophilic and/or hydrophobic polymers that are capable of controlling or retarding the release of the peptide or protein from a solid oral dosage form of the present invention. Suitable rate controlling polymer materials include those selected from the group consisting of hydroxyalkyl cellulose such as hydroxypropyl cellulose and hydroxypropyl methyl cellulose; poly(ethylene) oxide; alkyl cellulose such as ethyl cellulose and methyl cellulose; carboxymethyl cellulose; hydrophilic cellulose derivatives; polyethylene glycol; polyvinylpyrrolidone; cellulose acetate; cellulose acetate butyrate; cellulose acetate phthalate; cellulose acetate trimellitate; polyvinyl acetate phthalate; hydroxypropylmethyl cellulose phthalate; hydroxypropylmethyl cellulose acetate succinate; polyvinyl acetaldiethylamino acetate; poly(alkylmethacrylate) and poly (vinyl acetate). Other suitable hydrophobic polymers include polymers and/or copolymers derived from acrylic or methacrylic acid and their respective esters, zein, waxes, shellac and hydrogenated vegetable oils. Particularly useful in the practice of the present invention are poly acrylic acid, poly acrylate, poly methacrylic acid and poly methacrylate polymers such as those sold under the Eudragit tradename (Rohm GmbH, Darmstadt, Germany) specifically Eudragit® L, Eudragit® S, Eudragit® RL, and Eudragit® RS coating materials and mixtures thereof. Some of these polymers can be used as delayed release polymers to control the site where the drug is released. They include poly methacrylate polymers such as those sold under the Eudragit tradename (Rohm GmbH, Darmstadt, Germany) specifically Eudragit® L, Eudragit® S, Eudragit® RL, and Eudragit® RS coating materials and mixtures thereof.

Two important factors have been identified by the investigators for maximizing the bioavailability of the selective factor Xa inhibitor after oral administration of the pharmaceutical compositions described herein. The first is that the selective factor Xa inhibitor and the enhancer should be concurrently released at a substantially similar rate after the pharmaceutical composition enters the intestine of a subject. The second is that this release should occur rapidly. As a result of these two important factors, the interaction between the enhancer and the selective factor Xa inhibitor in the gastrointestinal tract may be maximized, which results in the most favorably improved bioavailability of the selective factor Xa inhibitor. The improved bioavailability allows the use of lower doses than previously needed and/or achievement of more effective treatment for the same dose. The investigators of the present application also observed that the relative in vivo performance of tablets containing an active pharmaceutical substance and the enhancer may be predicted by measuring the dissolution rate and/or disintegration rate for the active pharmaceutical substance and the enhancer from the dosage form in vitro.

As used herein, the term “rapid release rate” is defined as an in vitro dissolution of at least 80% of the selective factor Xa inhibitor and the enhancer from a dosage form without coating in 30 minutes. In other embodiments, the term “rapid release rate” is defined as an in vitro dissolution of at least 80% of the selective factor Xa inhibitor and the enhancer from a dosage form with a coating (e.g., an enteric coating or other type of delayed release or sustained release coating) in 40 minutes. The in vitro dissolution rate is determined by carrying out a dissolution assay in 900 mL pH 6.8 phosphate buffer at 37° C. with a USP Paddle Apparatus at 50 rpm. When the dosage form includes an enteric coating, the dissolution assay includes a preliminary step of acid treatment (2 hrs in 0.1 N HCl). The specific parameters for the dissolution assay are shown in Table 1. The term “dosage form without coating” refers to a dosage form comprising, consisting essentially of, or consisting of the pharmaceutical composition of the invention in the absence of any type of coating on the dosage form that would modulate the rate of release of the components of the dosage form (e.g., a delayed release or sustained release coating). In one embodiment, the dosage form is a tablet. Alternatively, the rapid release rate is defined as an in vitro dissolution of at least 90% of the selective factor Xa inhibitor and the enhancer from a dosage form without coating in 35 minutes. In another embodiment, the rapid release rate is defined as an in vitro dissolution of at least 90% of the selective factor Xa inhibitor and the enhancer from a dosage form with a coating in 45 minutes.

TABLE 1

| Acid Stage | 0.1N HCl |
|---|---|
| Apparatus | USP Type II (rotating paddles) |
| Rotations | 50 RPM |
| Dissolution Media | 0.1N HCl |
| Volume of Media | 900 ml |
| Temperature | 37.0° C. ± 0.5° C. |
| Time points | 120 minutes |
| Buffer Stage | Phosphate Buffer pH 6.8 |
| Apparatus | USP Type II (rotating paddles) |
| Rotations | 50 RPM |
| Dissolution Media | pH 6.8 Phosphate Buffer |
| Volume of Media | 900 ml |
| Temperature | 37.0° C. ± 0.5° C. |
| Time points | As appropriate |

As used herein, the term “substantially similar release” is defined as a ratio of the time for a percentage of the selective factor Xa inhibitor to be released from a dosage form without coating to the time for the same percentage of the enhancer to be released in the range of about 1.3 to about 0.7. In other embodiments, the term “substantially similar release” is defined as a ratio of the time for a percentage of the selective factor Xa inhibitor to be released from a dosage form with a coating (e.g., an enteric coating or other type of delayed release or sustained release coating) to the time for the same percentage of the enhancer to be released in the range of about 1.3 to about 0.7. In order to be considered a substantially similar release, the ratio must be in the range of about 1.3 to about 0.7 for at least 3 different percentages, e.g., at least 4, 5, 6, 7, or 8 different percentages. The in vitro dissolution rate is determined by carrying out a dissolution assay in 900 mL pH 6.8 phosphate buffer at 37° C. with a USP Paddle Apparatus at 50 rpm. When the dosage form includes an enteric coating, the dissolution assay includes a preliminary step of acid treatment (2 hrs in 0.1 N HCl). For example, if fondaparinux (selective factor Xa inhibitor) has a dissolution of 80% in about 20 minutes, sodium caprate (enhancer) must have a dissolution of 80% in the range of about 14 minutes to 26 minutes to be substantially similar. In one embodiment, the ratio is in the range of about 1.1 to about 0.9. For example, if fondaparinux (selective factor Xa inhibitor) has a dissolution of 80% in about 20 minutes, sodium caprate (enhancer) must have a dissolution of 80% in the range of about 18 minutes to about 22 minutes.

In one embodiment, the selective factor Xa inhibitor and the enhancer in a dosage form without coating have a substantially similar dissolution of at least about 95% in less than about 40 minutes in pH 6.8 phosphate buffer at 37° C. In another embodiment, the selective factor Xa inhibitor and the enhancer in a dosage form without coating have a substantially similar dissolution of at least about 95% in less than about 30 minutes in pH 6.8 phosphate buffer at 37° C. Further, in one embodiment, the selective factor Xa inhibitor and the enhancer in a dosage form without coating have a substantially similar dissolution of at least about 80% in less than about 20 minutes in pH 6.8 phosphate buffer at 37° C. In another embodiment, the selective factor Xa inhibitor and the enhancer in a dosage form without coating have a substantially similar dissolution of at least about 80% in less than about 18 minutes in pH 6.8 phosphate buffer at 37° C. In further embodiments, these dissolution rates are met with a coated dosage form.

Alternatively, the dissolution profile of the selective factor Xa inhibitor and the enhancer may also be compared using f1 and f2 values. Moore and Flanner (Pharm. Tech. 20(6): 64-74, 1996) proposed a model independent mathematical approach to compare the dissolution profile of two components using two factors, f1 and f2, as shown in the following formula.

f1={[Sₜ₌₁ⁿ(Rₜ−Tₜ)]/[Sₜ₌₁ⁿRₜ]}·100

f2=50·log {[1+(1/n)Sₜ₌₁ⁿ(Rₜ−Tₜ)²]^{-0.5}·100}

Here Rₜ and Tₜ are the cumulative percentage dissolved at each of the selected n time points of the reference and test product respectively. Relative standard deviation (RSD or RSD) is the absolute value of the coefficient of variation, often expressed as a percentage. The formula for calculating the % RSD may be described as: % Relative standard deviation=((standard deviation of array X)/(mean of array X))×100; X is the number of samples taken for each time point. The factor f1 is proportional to the average difference between the two profiles, whereas factor 12 is inversely proportional to the average squared difference between the two profiles, with emphasis on the larger difference among all the time points. The factor f2 measures the similarity between the two profiles. Because of the nature of the measurement, f1 is described as a difference factor, and f2 as a similarity factor.

When the two dissolution profiles are identical, f1=0 and f2=100. An average difference of 10% at all measured time points results in a f2 value of 50. The FDA has set a public standard of f2 value between 50-100 to indicate similarity between dissolution profiles of two tablets. It is generally accepted that an f1 value of less than 15 indicates similarity.

The data contained herein allows one to define a set of data inclusion criteria that are appropriate to determine whether a dosage form releases the selective factor Xa inhibitor rapidly enough and in sufficient conjunction with the enhancer to allow appropriate maximization of the effect of the enhancer. The following criteria apply: (1) at least 6 tablets should be used for each profile determination; (2) the mean dissolution values can be used to estimate the similarity factors (to use mean data, the % coefficient of variation at the earliest point should not be more than 30% and at other time points should not be more than 20%; and (3) at least 4 dissolution values must be used in the calculation, none of which can be 0, and only one of which can be greater than 85% dissolution.

The same time points must be used for both the selective factor Xa inhibitor and the enhancer. Therefore it may not be possible to satisfy all the criteria for dissolution of both the enhancer and selective factor Xa inhibitor simultaneously. In one example, for a formulation where non-co-release occurs it may be necessary for one of the profiles (for the faster component) to have more than one value above 85%. In another example, for a formulation where non-co-release occurs it may not be possible for both profiles to satisfy the % RSD requirements at the same time points, due to significantly lower percent dissolved of one component over the other at that time point.

The Moore and Flanner model independent mathematical approach has been adapted to compare the dissolution profile of enhancer and selective factor Xa inhibitor and define co-release. Substantially similar co-release is defined herein as a f1 value of less than 15. For quality control purposes for comparisons of tablets containing the same active ingredient with different formulations, a f1 value of less than 15 is generally accepted to indicate similarity.

A f2 value of 50-100 is defined herein to indicate substantially similar co-release of the selective factor Xa inhibitor and enhancer. The inventors are not aware of anyone using this sort of approach to optimize and ensure that an oral absorption enhancer is appropriately formulated with an active drug substance to assure appropriate enhancer performance.

In some embodiments, for f1 and f2 analysis, the number of time points may be 4, 5, 6, 7, 8, or 9 or more. It is understood by one skilled in the art that, even with the criteria defined above, f1 and f2 values may be manipulated by changing the number and/or time intervals of sample points, their location on the dissolution curve, and other variants. Thus, the f1 and f2 calculations are tools to compare the dissolution profile of different formulations and demonstrate the properties of the pharmaceutical compositions described herein. In addition, the f1 and f2 calculations may also be used as tools to compare enhancer and selective factor Xa inhibitor release within one formulation. The scope of the invention should not be limited to the exact value of f1 and f2.

In one embodiment of the invention, the f1 value for the dissolution profile of the enhancer and the selective factor Xa inhibitor is less than about 25, e.g., less than about 20, 15, or 5. In other embodiments of the invention, the f2 value for the dissolution profile of the enhancer and the selective factor Xa inhibitor is at least about 50, e.g., at least about 55, 60, 65, 70, 75, 80, 85, 90 or 95.

For instantly soluble pharmaceutical compositions, the disintegration rate may predict the dissolution behavior because the disintegration of the dosage form of the pharmaceutical composition may be the rate-limiting step to dissolution. The disintegration test used to test the dosage form of the pharmaceutical compositions described herein is carried out as described in the EP 2.9.1 monograph Disintegration of Tablets and Capsules for uncoated tablets. The compendia recommendation is to use water. The temperature for the test is 37° C. According to some aspects of the present invention, the pharmaceutical compositions described herein provide a relatively fast disintegration rate. In one embodiment, the pharmaceutical composition in a dosage form without coating has a disintegration time of less than about 15 minutes at 37° C. In another embodiment, the pharmaceutical composition in a dosage form without coating has a disintegration time of less than about 10 minutes at 37° C.

Another aspect of the present invention provides a method of treating or preventing a medical condition, comprising administering to a patient suffering from said condition, a therapeutically effective amount of a selective factor Xa inhibitor or a pharmaceutically acceptable salt thereof together with an enhancer, wherein the enhancer is a medium chain fatty acid or a salt, ester, ether, or derivative of a medium chain fatty acid and has a carbon chain length of from 4 to 20 carbon atoms. In some embodiments, the medical condition is a thromboembolic condition, e.g., thrombophlebitis, phlebothrombosis, venous thrombosis, deep vein thrombosis, portal vein thrombosis, renal vein thrombosis, jugular vein thrombosis, Budd-Chiari syndrome, Paget-Schroetter disease, cerebral venous sinus thrombosis, pulmonary embolism, arterial thrombosis, stroke, myocardial infarction, hepatic artery thrombosis, arterial embolus, or any combination thereof.

Another aspect of the present invention provides use of the compositions of the invention for treatment of a medical condition, e.g., a thromboembolic condition. The invention also relates to compositions and pharmaceutical formulations as described herein for use in the treatment of a medical condition, e.g. a thromboembolic condition.

Another aspect of the present invention provides use of the compositions of the invention in the manufacture of a medicament for treatment of a medical condition, e.g., a thromboembolic condition.

Another aspect of the present invention provides a process for manufacturing a solid oral dosage form of a pharmaceutical composition comprising the steps of: a) blending a selective factor Xa inhibitor or a pharmaceutically acceptable salt thereof with an enhancer, and optionally auxiliary excipients to form a blend; wherein the enhancer is a medium chain fatty acid or a salt, ester, ether, or derivative of a medium chain fatty acid and has a carbon chain length of from 4 to 20 carbon atoms; and b) forming a solid oral dosage from the blend by i) directly compressing the blend to form the solid oral dosage form, or ii) granulating the blend to form a granulate for incorporation into the solid oral dosage form, or iii) spray drying the blend to form a multiparticulate for incorporation into the solid oral dosage form. In some embodiments, the selective factor Xa inhibitor and the enhancer are blended in a ratio of from 1:100000 to 10:1 (inhibitor:enhancer)), e.g., from 1:1000 to 5:1, e.g., from 1:300 to 1:1.

Another aspect of the present invention provides a pharmaceutical composition of a selective factor Xa inhibitor or a pharmaceutically acceptable salt thereof for oral administration, the composition comprising, consisting essentially of, or consisting of a stable, transparent drug delivery composition. The stable, transparent drug delivery composition comprises, consists essentially of, or consists of (a) from about 1 to about 80 weight percent of a pharmaceutically acceptable oil; (b) from about 3 to about 98 weight percent (e.g., from about 3 to about 96.5 weight percent) surfactants; (c) from about 2 to about 60 weight percent polyethylene glycol; (d) from about 0.5 to about 15 weight percent water; and (e) a selective factor Xa inhibitor or a pharmaceutically acceptable salt thereof; wherein the ratio of the polyethylene glycol to water is at least 2:1. In certain embodiments, the composition does not contain a mixture of cholesterol and phospholipid. In some embodiments, the pharmaceutical composition of a selective factor Xa inhibitor further comprises an enhancer, wherein the enhancer is a medium chain fatty acid or a salt, ester, ether, or derivative of a medium chain fatty acid and has a carbon chain length of from 4 to 20 carbon atoms. In some embodiments, the pharmaceutical composition described above can provide a stable and reproducible bioavailability of the selective factor Xa inhibitor. The stable drug delivery composition is further described in U.S. Pat. No. 5,707,648, which is incorporated by reference in its entirety.

“Pharmaceutically acceptable oils” include oils accepted in the food or pharmaceutical industry, e.g., triesters of glycerol having from 9 to 83, e.g., 21-60, e.g., 21-45 carbon atoms. The triglycerides are further defined as short chain triglycerides having 9-15 carbon atoms, medium chain triglycerides having 21-45 carbon atoms, and long chain triglycerides having above 45 carbon atoms. Medium chain triglycerides are preferred. Examples of glycerol triesters include natural, edible oils such as canola, corn, olive, sunflower and coconut oils, triacetin, the decanoic acid esters, and chemically-synthesized oils such as 1-oleyl-2,3-diacetyl glycerol. Commercially available triglyceride oils, both natural and chemically-synthesized, are available from Karlshamns Lipid Specialties, U.S.A. as the Captex® series, and from Huls America Inc. as the Miglyol series. Other suitable oils include diesters of propylene glycol having from 7 to 55, e.g., 15-40 carbon atoms, e.g., propylene glycol esters of capric and caprylic acids, and mixtures thereof, having from 19 to 23 carbon atoms. The diesters of propylene glycols are further defined as short chain having from 7-11 carbon atoms, medium chain having from 15-31 carbon atoms, and long chain having above 31 carbon atoms. Preferred propylene glycol diesters are the medium chain oils. Diesters of propylene glycols include propylene glycol esters of capric acid, caprylic acid, and mixtures thereof such as Captex® 200, and Captex® 800 (Karlshamns Lipid Specialties, Columbus, Ohio) and other ester groups as described above for glycerol.

Surfactants (surface active agents) which may be employed in the compositions include both ionic agents, i.e., cationic, anionic or zwitterionic, and non-ionic agents, or mixtures thereof. Examples of cationic surfactants include cetyldimethylethylammonium bromide, cetylpyridinium chloride and other salts of these surfactants. Short chain monohydroxyl alcohols, such as C₁ to C₆ alcohols, are preferably not employed as surfactants in these systems due to toxicity factors, thus the compositions are substantially free of such short chain monohydroxyl alcohols. Various surfactants also have permeation enhancement properties.

Examples of anionic surfactants include C₈₋₃₂ fatty acids and salts thereof, e.g., C₈₋₁₂, e.g., C₈; cholic acid and derivatives thereof such as deoxycholate, and its salts, ursodeoxycholic acid, and taurocholic acid; C₈₋₅₆ diesters of tartaric acid; phospholipids such as phosphatidic acid and phosphatidyl serine; C₅₋₂₉ monoesters of lactic acid; C₈₋₂₀ sulfonates, including alkyl-, olefin-, and alkylaryl derivatives; tridecyl- and dodecylbenzene sulfonic acids; and C₅₋₃₃ sarcosine and betaine derivatives.

Zwitterionics include such phospholipids as lecithin, phosphatidylethanolamine, and sphingomyelins.

Among the non-ionic surfactants which may be employed are ethoxylated castor oil; C₅₋₂₉ mono-glycerides and ethoxylated derivatives thereof; C₁₅₋₆₀ diglycerides and polyoxyethylene derivatives thereof having 1 to 90 POE groups; C₁₀₋₄₀ esters (10-40 carbon atoms in the alcohol) of long chain fatty acids (fatty acids having 16 carbon atoms and above); C₁₀₋₄₀ alcohols; sterols such as cholesterol, ergosterol, and C₂₋₂₄ esters thereof; C₈₋₉₆ ethoxylated fatty esters; C₁₄₋₁₃₀ sucrose fatty esters; and C₂₀₋₁₃₀ sorbitol and sorbitan monoesters, diesters, and triesters, and polyoxyethylene (POE) derivatives thereof having 1 to 90 POE groups, e.g., polyoxyethylene sorbitan monooleate, sorbitol hexaoleate POE (50).

Low HLB surfactants include C₉ to C₁₃ monoglycerides, C₁₉ to C₂₅ diglycerides of mono and poly unsaturated fatty acids, C₁₅ to C₂₃ diglycerides, and C₃₅ to C₄₇ diglycerides of mono and poly unsaturated fatty acids. Preferred low HLB surfactants are those containing at least about 80 percent by weight, e.g., at least about 90 percent by weight, e.g., at least about 95 percent by weight, of a monoglyceride or diglyceride containing C₆, C₇, C₈, C₉, or C₁₀ fatty acid functionalities, or mixtures thereof, e.g., a C₉, C₁₁, or C₁₃ monoglyceride or mixtures thereof, e.g., a C₁₁ or C₁₃ monoglyceride or mixtures thereof. Commercial examples of these surfactants include Imwitor 308, manufactured by Huls America, Inc., having about 80-90% wt. C₁₁ monoglycerides; and Glycerol Monocaprylin, manufactured by Sigma Chemicals as 1-monooctanoyl-rac-glycerol having about 99% wt. C₁₁ monoglycerides, and Glycerol Monocaprate, manufactured as 1-monodecanoyl-rac-glycerol by Sigma Chemicals, having about 99% wt. C₁₃ monoglycerides. In certain embodiments, the low HLB surfactant, or mixture of low HLB surfactants, will be only the above recited monoglycerides having a purity of at least about 80 weight percent.

High HLB surfactants include the sorbitan surfactants, e.g., those having an HLB of from about 13 to about 17. Such surfactants include POE (20) sorbitan monooleate, monostearate, monopalmitate, and monolaurate sold commercially as Tween 80, 60, 40, and 20, respectively, by ICI Inc., and POE (4) sorbitan monolaurate sold commercially as Tween 21 by ICI. Other high HLB surfactants include ethoxylated castor oil surfactants, e.g., those having an HLB of from about 12 to about 20, such as Cremophor EL, RH-40, and RH-60 and the Pluronic F-series sold by BASF Inc. Potassium oleate is also preferred as a high HLB surfactant.

The low HLB surfactant may be present in the composition in an amount of from about 1 to about 40, e.g., from about 5 to about 30, e.g., from about 10 to about 30 weight percent, e.g., from 20 to 30 weight percent. The high HLB surfactant may be present in the composition in an amount of from about 2 to about 60, e.g., from about 5 to about 50, e.g., from about 10 to about 40 weight percent.

One aspect of the present invention describes a pharmaceutical composition of a selective factor Xa inhibitor or a pharmaceutically acceptable salt thereof for oral administration, the composition comprising, consisting essentially of, or consisting of an emulsion composition, wherein an internal phase of the emulsion composition contains a therapeutically effective amount of a selective factor Xa inhibitor or a pharmaceutically acceptable salt thereof; and the internal phase comprises or consists essentially of a polar, nonaqueous oxygen-containing, pharmaceutically acceptable liquid selected from the group consisting of C₂-C₃₀ polyhydric alcohols, poly(ethylene or propylene) glycols with 4-200 repeating units, C₂-C₃₀ ester derivatives thereof and C₁-C₅ ether derivatives thereof. Examples of such materials include glycerin, propylene glycol, polyethylene glycol 200, 400, 600, 1500, 4000 and 6000 with the number correlating approximately with the number of repeating units and ranging from 4 to 200, ethylene glycol dimethyl ether, tetraethylene glycol dimethyl ether, triacetin, medium chain (C₆-C₁₀) triglycerides such as tricaprylin (caprylic acid ester of glycerol, and propylene glycol C₈ diester (Captex 200). In certain embodiments the internal phase is a C₂-C₁₀ polyhydric alcohol, a polyethylene glycol with n=4−80, or the methyl or ethyl ethers thereof. The emulsion may also contain lecithin as an emulsifier or surfactant. Egg or soya lecithin is suitable. The continuous phase of the emulsion may be a lower alkyl ester of a C₈-C₂₂ fatty acid such as ethyl palmitate or a triglyceride. The alkyl may be C₁₋₅, e.g., C₁₋₃. In some embodiments, the pharmaceutical composition of a selective factor Xa inhibitor further comprises an enhancer, wherein the enhancer is a medium chain fatty acid or a salt, ester, ether, or derivative of a medium chain fatty acid and has a carbon chain length of from 4 to 20 carbon atoms. In some embodiments, the pharmaceutical composition described above can provide a stable and reproducible bioavailability of the selective factor Xa inhibitor. The emulsion composition is further described in U.S. Pat. No. 5,110,606, which is incorporated by reference in its entirety.

Further aspects of the present invention relate to microemulsion compositions containing a selective factor Xa inhibitor. In certain embodiments the microemulsions are water-in-oil microemulsions. In some embodiments the selective factor Xa inhibitor is present in an internal dispersed aqueous phase. In one aspect, the composition is a water-in-oil microemulsion comprising an internally dispersed aqueous phase containing the selective factor Xa inhibitor; a pharmaceutically acceptable oil; and a surfactant component (which may comprise a mixture of surfactants) having a HLB value of 7-14. The pharmaceutically acceptable oil is as defined above, but in some aspects is selected from the group consisting of mono and di-esters of propylene glycol having from 15 to 40 carbon atoms, C₉₋₈₃ triglycerides, C₇₋₅₅ mono- or di-esters of propylene glycol, or mixtures thereof. The microemulsions may further comprise an enhancer, e.g., a medium chain fatty acid salt, an ester, an ether, or a derivative of a medium chain fatty acid and has a carbon chain length of from 4 to 20 carbon atoms.

Preferably the internal aqueous phase of the microemulsion comprises up to about 60 volume percent, e.g., up to about 20 volume percent of the microemulsion composition. In some embodiments the pharmaceutically acceptable oil component of the microemulsion comprises from about 5 to about 99 volume percent, e.g., from about 30 to about 99 volume percent or about 5 to about 90 volume percent of the microemulsion composition. In one embodiment the surfactant component of the microemulsion comprises from about 1 to about 70 volume percent of the microemulsion composition.

Another aspect of the present invention provides a pharmaceutical composition of a selective factor Xa inhibitor or a pharmaceutically acceptable salt thereof for oral administration which is a water-in-oil microemulsion composition, wherein the microemulsion composition converts to an oil-in-water emulsion by the addition of water and the microemulsion composition comprises, consists essentially of, or consists of (a) up to about 20 volume percent of an internal dispersed aqueous phase containing a therapeutically effective amount of a selective factor Xa inhibitor or a pharmaceutically acceptable salt thereof; (b) from about 30 to about 99 volume percent of a continuous oil phase comprising mono and di-esters of propylene glycol having from 15 to 40 carbon atoms; and (c) from about 1 to about 70 volume percent of a surfactant or mixture of surfactants, wherein the surfactant or surfactant mixture has a HLB value of from 7 to 14. In some embodiments, the pharmaceutical composition of a selective factor Xa inhibitor further comprises an enhancer, wherein the enhancer is a medium chain fatty acid salt, an ester, an ether, or a derivative of a medium chain fatty acid and has a carbon chain length of from 4 to 20 carbon atoms. In some embodiments, the pharmaceutical composition described above can provide a stable and reproducible bioavailability of the selective factor Xa inhibitor. The microemulsion composition is further described in U.S. Pat. No. 5,444,041, which is incorporated by reference in its entirety.

One aspect of the present invention describes a pharmaceutical composition of a selective factor Xa inhibitor or a pharmaceutically acceptable salt thereof for oral administration which is a water-in-oil microemulsion composition, wherein the microemulsion composition converts to an oil-in-water emulsion by the addition of water and the microemulsion composition comprises, consists essentially of, or consists of (a) up to about 60 volume percent, based upon the total volume of the microemulsion, of an internally dispersed aqueous phase containing a therapeutically effective amount of a selective factor Xa inhibitor or a pharmaceutically acceptable salt thereof; (b) from about 5 to about 90 volume percent of a continuous oil phase comprising at least one pharmaceutically acceptable oil; and (3) from about 1 to about 70 volume percent of a surfactant or mixture of surfactants, wherein the surfactant or surfactant mixture has a HLB value of from 7 to 14. In some embodiments, the pharmaceutical composition of a selective factor Xa inhibitor further comprises an enhancer, wherein the enhancer is a medium chain fatty acid salt, an ester, an ether, or a derivative of a medium chain fatty acid and has a carbon chain length of from 4 to 20 carbon atoms. In some embodiments, the pharmaceutical composition described above can provide a stable and reproducible bioavailability of the selective factor Xa inhibitor. The microemulsion composition is further described in U.S. Pat. No. 5,646,109, which is incorporated by reference in its entirety.

Another aspect of the present invention provides a pharmaceutical composition of a selective factor Xa inhibitor or a pharmaceutically acceptable salt thereof for oral administration which is a water-in-oil microemulsion composition, wherein the microemulsion composition comprises, consists essentially of, or consists of (a) from about 5 to about 99 volume percent of an oil phase comprising at least one pharmaceutically acceptable oil; (b) up to about 60 volume percent of an aqueous phase comprising water; (c) a therapeutically effective amount of a selective factor Xa inhibitor or a pharmaceutically acceptable salt thereof; (d) from about 1 to about 70 volume percent of a mixture of surfactants having a combined HLB value of from about 7 to about 14 comprising (i) a low HLB surfactant having an HLB below 8, said low HLB surfactant being at least 40 percent by weight of a C₉ monoglyceride, C₁₀ monoglyceride, C₁₁ monoglyceride, C₁₂ monoglyceride, or C₁₃ monoglyceride, and (ii) at least one surfactant having a HLB value above about 8. In some embodiments, the pharmaceutical composition of a selective factor Xa inhibitor further comprises an enhancer, wherein the enhancer is a medium chain fatty acid salt, an ester, an ether, or a derivative of a medium chain fatty acid and has a carbon chain length of from 4 to 20 carbon atoms. In some embodiments, the pharmaceutical composition described above can provide a stable and reproducible bioavailability of the selective factor Xa inhibitor. The microemulsion composition is further described in U.S. Pat. No. 5,688,761, which is incorporated by reference in its entirety.

One aspect of the present invention provides a pharmaceutical composition of a selective factor Xa inhibitor or a pharmaceutically acceptable salt thereof for oral administration which is a water-in-oil microemulsion composition, wherein the microemulsion composition comprises, consists essentially of, or consists of (a) up to about 60 volume percent of an internal dispersed aqueous phase containing a therapeutically effective amount of a selective factor Xa inhibitor or a pharmaceutically acceptable salt thereof; (b) from about 5 to about 99 volume percent of a continuous oil phase comprising at least one pharmaceutically acceptable oil comprising a C₉₋₈₃ triglyceride, a C₇₋₅₅ mono- and di-ester of propylene glycol, or mixtures thereof; and (c) from about 1 to about 70 volume percent of a surfactant or surfactant mixture comprising a C₈ fatty acid salt, wherein the surfactant or surfactant mixture has a HLB value of at least 7. In some embodiments, the pharmaceutical composition of a selective factor Xa inhibitor further comprises an enhancer, wherein the enhancer is a medium chain fatty acid salt, an ester, an ether, or a derivative of a medium chain fatty acid and has a carbon chain length of from 4 to 20 carbon atoms. In some embodiments, the pharmaceutical composition described above can provide a stable and reproducible bioavailability of the selective factor Xa inhibitor. The microemulsion composition is further described in U.S. Pat. No. 5,633,226, which is incorporated by reference in its entirety.

In some embodiments, the pharmaceutical composition described above can provide a reproducible and predictable bioavailability, Cₘₐₓ, or other pharmacokinetic characteristic of the selective factor Xa inhibitor.

In some embodiments, in the pharmaceutical compositions described above, the selective factor Xa inhibitor is fondaparinux or a pharmaceutically acceptable salt thereof.

Another aspect of the present invention provides a method for obtaining a reproducible pharmacokinetic characteristic (e.g., bioavailability, Cₘₐₓ, AUC, etc.) of a selective factor Xa inhibitor in a subject after oral administration, comprising orally administering a pharmaceutical composition of the present invention to said subject. In certain embodiments, the composition comprises, consists essentially of, or consists of (a) a therapeutically effective amount of a selective factor Xa inhibitor or a pharmaceutically acceptable salt thereof and (b) an enhancer, wherein the enhancer is a medium chain fatty acid or a salt, ester, ether, or derivative of a medium chain fatty acid and has a carbon chain length of from 4 to 20 carbon atoms.

In some embodiments, the pharmaceutical composition comprises, consists essentially of, or consists of: (a) from about 1 to about 80 weight percent of a pharmaceutically acceptable oil; (b) from about 3 to about 98 weight percent (e.g., from about 3 to about 96.5 weight percent) surface active agents; (c) from about 2 to about 60 weight percent polyethylene glycol; and (d) from about 0.5 to about 15 weight percent water; wherein the ratio of the polyethylene glycol to water is at least 2:1.

In some embodiments, the pharmaceutical composition is in a form of a transparent drug delivery composition, wherein the drug delivery composition comprises, consists essentially of, or consists of: (a) from about 1 to about 80 weight percent of a pharmaceutically acceptable oil; (b) from about 3 to about 98 weight percent (e.g., from about 3 to about 96.5 weight percent) surface active agents; (c) from about 2 to about 60 weight percent polyethylene glycol; and (d) from about 0.5 to about 15 weight percent water; wherein the ratio of the polyethylene glycol to water is at least 2:1.

In some embodiments, the pharmaceutical composition is an emulsion composition, wherein an internal phase of the emulsion composition contains a therapeutically effective amount of a selective factor Xa inhibitor or a pharmaceutically acceptable salt thereof; and the internal phase comprises or consists essentially of a polar, nonaqueous oxygen-containing, pharmaceutically acceptable liquid selected from the group consisting of C₂-C₃₀ polyhydric alcohols, poly(ethylene or propylene) glycols with 4-200 repeating units, C₂-C₃₀ ester derivatives thereof, and C₁-C₅ ether derivatives thereof.

In some embodiments, the pharmaceutical composition is a water-in-oil microemulsion composition, wherein the microemulsion composition converts to an oil-in-water emulsion by the addition of water and the microemulsion composition comprises, consists essentially of, or consists of (a) up to about 20 volume percent of an internal dispersed aqueous phase containing a therapeutically effective amount of selective factor Xa inhibitor or a pharmaceutically acceptable salt thereof, (b) from about 30 to about 99 volume percent of a continuous oil phase comprising mono and di-esters of propylene glycol having from 15 to 40 carbon atoms, and (c) from about 1 to about 70 volume percent of a surfactant or mixture of surfactants, wherein the surfactant or surfactant mixture has a HLB value of from 7 to 14.

In some embodiments, the pharmaceutical composition is a water-in-oil microemulsion composition, wherein the microemulsion composition converts to an oil-in-water emulsion by the addition of water and the microemulsion composition comprises, consists essentially of, or consists of (a) up to about 60 volume percent, based upon the total volume of the microemulsion, of an internally dispersed aqueous phase containing a therapeutically effective amount of a selective factor Xa inhibitor or a pharmaceutically acceptable salt thereof, (b) from about 5 to about 90 volume percent of a continuous oil phase comprising at least one pharmaceutically acceptable oil; and (c) from about 1 to about 70 volume percent of a surfactant or mixture of surfactants, wherein the surfactant or surfactant mixture has a HLB value of from 7 to 14.

In some embodiments, the pharmaceutical composition is a water-in-oil microemulsion composition, wherein the microemulsion composition comprises, consists essentially of, or consists of (a) from about 5 to about 99 volume percent of an oil phase comprising at least one pharmaceutically acceptable oil; (b) up to about 60 volume percent of an aqueous phase comprising water; (c) a therapeutically effective amount of a selective factor Xa inhibitor or a pharmaceutically acceptable salt thereof; (d) from about 1 to about 70 volume percent of a mixture of surfactants having a combined HLB value of from about 7 to about 14 comprising (i) a low HLB surfactant having a HLB below 8, said low HLB surfactant being at least 40 percent by weight of a C₉ monoglyceride, C₁₀ monoglyceride, C₁₁ monoglyceride, C₁₂ monoglyceride, or C₁₃ monoglyceride, and (ii) at least one surfactant having a HLB value above about 8.

In some embodiments, the pharmaceutical composition is a water-in-oil microemulsion composition, wherein the microemulsion composition comprises, consists essentially of, or consists of (a) up to about 60 volume percent of an internal dispersed aqueous phase containing a therapeutically effective amount of a selective factor Xa inhibitor or a pharmaceutically acceptable salt thereof; (b) from about 5 to about 99 volume percent of a continuous oil phase comprising at least one pharmaceutically acceptable oil comprising a C₉₋₈₃ triglyceride, a C₇₋₅₅ mono- and di-ester of propylene glycol, or mixtures thereof; and (c) from about 1 to about 70 volume percent of a surfactant or surfactant mixture comprising a C₈ fatty acid salt, wherein the surfactant or surfactant mixture has a HLB value of at least 7.

One aspect of the present invention provides a method for obtaining a reproducible pharmacokinetic characteristic (e.g., bioavailability, Cₘₐₓ, AUC, etc.) of a selective factor Xa inhibitor in a subject after oral administration, comprising orally administering a pharmaceutical composition of the invention to said subject.

In some embodiments of the invention, the pharmaceutical compositions of the invention provide a bioavailability of the selective factor Xa inhibitor or a pharmaceutically acceptable salt thereof of at least about 5% when orally administered to a human subject, e.g., at least about 5, 6, 7, 8, 9, 10, 11, 12, 13, 14, 15, 16, 17, 18, 19, 20, 21, 22, 23, 24, or 25% or more. In other embodiments of the invention, the pharmaceutical compositions of the invention provide a bioavailability of the selective factor Xa inhibitor or a pharmaceutically acceptable salt thereof of at least about 5% when intraduodenally administered to a beagle dog, e.g., at least about 5, 6, 7, 8, 9, 10, 11, 12, 13, 14, 15, 16, 17, 18, 19, 20, 21, 22, 23, 24, or 25% or more. In other embodiments of the invention, the pharmaceutical compositions of the invention provide a bioavailability of the selective factor Xa inhibitor or a pharmaceutically acceptable salt thereof of at least about 5% when orally administered to a beagle dog, e.g., at least about 5, 6, 7, 8, 9, 10, 11, 12, 13, 14, 15, 16, 17, 18, 19, 20, 21, 22, 23, 24, or 25% or more.

In some embodiments, the invention encompasses any pharmaceutical composition (e.g., compositions equivalent to the compositions described herein) that provides a bioavailability of the selective factor Xa inhibitor or a pharmaceutically acceptable salt thereof of at least about 5% when orally administered to a human subject, e.g., at least about 5, 6, 7, 8, 9, 10, 11, 12, 13, 14, 15, 16, 17, 18, 19, 20, 21, 22, 23, 24, or 25% or more. In other embodiments, the invention encompasses any pharmaceutical composition (e.g., compositions equivalent to the compositions described herein) that provides a bioavailability of the selective factor Xa inhibitor or a pharmaceutically acceptable salt thereof of at least about 5% when intraduodenally administered to a beagle dog, e.g., at least about 5, 6, 7, 8, 9, 10, 11, 12, 13, 14, 15, 16, 17, 18, 19, 20, 21, 22, 23, 24, or 25% or more. In other embodiments, the invention encompasses any pharmaceutical composition (e.g., compositions equivalent to the compositions described herein) that provides a bioavailability of the selective factor Xa inhibitor or a pharmaceutically acceptable salt thereof of at least about 5% when orally administered to a beagle dog, e.g., at least about 5, 6, 7, 8, 9, 10, 11, 12, 13, 14, 15, 16, 17, 18, 19, 20, 21, 22, 23, 24, or 25% or more.

In certain embodiments of the invention, the pharmaceutical compositions of the invention provide a reproducible bioavailability from subject to subject after oral administration, e.g., a decreased coefficient of variation (CV) relative to the CV of unenhanced compositions. In some embodiments, the CV for bioavailability is less than about 60%, e.g., less than about 60, 55, 50, 45, 40, 35, 30, 25, 20, 15% or less when orally administered to human subjects. In other embodiments, the CV for bioavailability is less than about 60%, e.g., less than about 60, 55, 50, 45, 40, 35, 30, 25, 20, 15% or less when intraduodenally administered to beagle dogs. In other embodiments, the CV for bioavailability is less than about 60%, e.g., less than about 60, 55, 50, 45, 40, 35, 30, 25, 20, 15% or less when orally administered to beagle dogs.

In certain embodiments of the invention, the pharmaceutical compositions of the invention provide a reproducible Cₘₐₓ from subject to subject after oral administration, e.g., a decreased coefficient of variation (CV) relative to the CV of unenhanced compositions. In some embodiments, the CV for Cₘₐₓ is less than about 70%, e.g., less than about 70, 65, 60, 55, 50, 45, 40, 35, 30, 25, 20, 15% or less when orally administered to human subjects. In other embodiments, the CV for Cₘₐₓ is less than about 70%, e.g., less than about 70, 65, 60, 55, 50, 45, 40, 35, 30, 25, 20, 15% or less when intraduodenally administered to beagle dogs. In other embodiments, the CV for Cₘₐₓ is less than about 70%, e.g., less than about 70, 65, 60, 55, 50, 45, 40, 35, 30, 25, 20, 15% or less when orally administered to beagle dogs.

In certain embodiments of the invention, the pharmaceutical compositions of the invention provide a reproducible AUC from subject to subject after oral administration, e.g., a decreased coefficient of variation (CV) relative to the CV of unenhanced compositions. In some embodiments, the CV for AUC is less than about 60%, e.g., less than about 60, 55, 50, 45, 40, 35, 30, 25, 20, 15% or less when orally administered to human subjects. In other embodiments, the CV for AUC is less than about 60%, e.g., less than about 60, 55, 50, 45, 40, 35, 30, 25, 20, 15% or less when intraduodenally administered to beagle dogs. In other embodiments, the CV for AUC is less than about 60%, e.g., less than about 60, 55, 50, 45, 40, 35, 30, 25, 20, 15% or less when orally administered to beagle dogs.

In some embodiments of the invention, the dose of selective factor Xa inhibitor administered to a subject is a dose sufficient to treat or prevent a medical condition. For example, the total dose administered to a subject can be in the range of about 2 to about 200 mg or more, e.g., about 2, 4, 6, 8, 10, 12.5, 15, 20, 25, 30, 35, 40, 45, 50, 55, 60, 65, 70, 75, 80, 85, 90, 95, 100, 120, 140, 160, 180, or 200 mg or more or any range therein. The dosage forms of the invention can comprise any convenient amount of selective factor Xa inhibitor, e.g., about 1, 2.5, 5, 7.5, 10, 12.5, 15, 20, 25, 30, 35, 40, 45, 50, 55, 60, 70, 75, 80, 85, 90, 95, 100, 125, 150, 175, or 200 mg or more.

In some embodiments, in the methods described above, the selective factor Xa inhibitor is fondaparinux or a pharmaceutically acceptable salt thereof.

The present invention will now be described in more detail with reference to the following examples. However, these examples are given for the purpose of illustration and are not to be construed as limiting the scope of the invention.

In the examples, it can be seen that the formulations of the present invention can achieve a bioavailability increase for fondaparinux of over 9% as compared to subcutaneous injection and in certain formulations, over a 16% bioavailability increase in achieved, even up to a 18.5% bioavailability increase. It can also be seen that the formulations of the present invention significantly increased the bioavailability of the drug (fondaparinux) and decreased the variability of absorption compared to an unenhanced formulation. Comparable pharmacokinetic profiles were obtained with most preferred fondaparinux formulations to that shown with the subcutaneous reference injection.

### Example 1

### Bioavailability of Oral Dosage Forms of Fondaparinux

The aim of the study was to determine the feasibility of preparing an oral dosage form of fondaparinux using Gastrointestinal Permeation Enhancement Technology (GIPET™) penetration enhancing technology. An intraduodenally cannulated dog model was used to determine fondaparinux bioavailability from solutions of fondaparinux in GIPET™ matrices administered directly into the duodena of beagle dogs. GIPET™ I technology involves the use of enteric coated tablets while GIPET™ II technology is microemulsion based within an enteric coated soft gel/hard capsule shell. Two GIPET™ I formulations (high and low) and two GIPET™ II formulations (Form I and Form II) were prepared with the components listed in Tables 2 and 3. An unenhanced solution was administered as a control, and a subcutaneous (s.c.) injection was administered as a reference dosage form.

TABLE 2

| | | Formulation | | | |
|---|---|---|---|---|---|
| | | Dose | | Dosing | |
| TI | Designation | fondaparinux | Formulation | Volume | Flush |
| Test | Reference | 1 mg | ARIXTRA ® | 0.2 mL | — |
| Item 1 | subcutaneous | | 2.5 mg/0.5 mL | | |
| | | | isotonic saline | | |
| Test | Reference | 5 mg | In water | 10 mL | 3 mL |
| Item 2 | Unenhanced | | | | |
| Test | GIPET ™ I | 5 mg | In solution | 10 mL | 3 mL |
| Item 3 | (High) | | containing | | |
| | | | 550 mg C10 | | |
| Test | GIPET ™ I | 5 mg | In solution | 10 mL | +3 mL |
| Item 4 | (Low) | | containing | | |
| | | | 275 mg C10 | | |
| Test | GIPET ™ II | 5 mg | In Capmul ® | 1 mL | 12 mL |
| Item 5 | Form I | | MCM based | | |
| | | | microemulsion | | |
| Test | GIPET ™ II | 5 mg | In Capmul ® | 1 mL | 12 mL |
| Item 6 | Form II | | MCM C10 | | |
| | | | based | | |
| | | | microemulsion | | |

TABLE 3

| | Capmul MCM | Capmul MCM |
|---|---|---|
| | Based | C10 Based |
| Raw Material | % | % |
| Captex ® 300 | 5.0 | 13.3 |
| (Glyceryl tricaprylate | | |
| caprate) | | |
| Capmul ® MCM | 41.9 | — |
| (Glyceryl caprylate caprate) | | |
| Capmul ® MCM C10 | — | 36.6 |
| (Glyceryl caprate) | | |
| Tween ® 80 | 21.3 | 21.3 |
| (Polyethylene oxide | | |
| sorbitan mono-oleate) | | |
| PEG 400 | 23.3 | 23.3 |
| Purified water | 8.0 | 5.0 |
| fondaparinux | 0.5 | 0.5 |

Five female beagle dogs had previously been surgically implanted with duodenal access ports (DAPs). Each DAP is connected to a cannula which is inserted into the duodenum. Blood samples were taken at appropriate intervals after administration of each test item and assayed for anti-Factor Xa activity.

The study consisted of six (6) test items: a reference dose of commercially available fondaparinux (subcutaneous injection), an unenhanced aqueous formulation, and four (4) GIPET™ liquid formulations. The test items were administered weekly as single bolus doses. Test item details are given in Table 2.

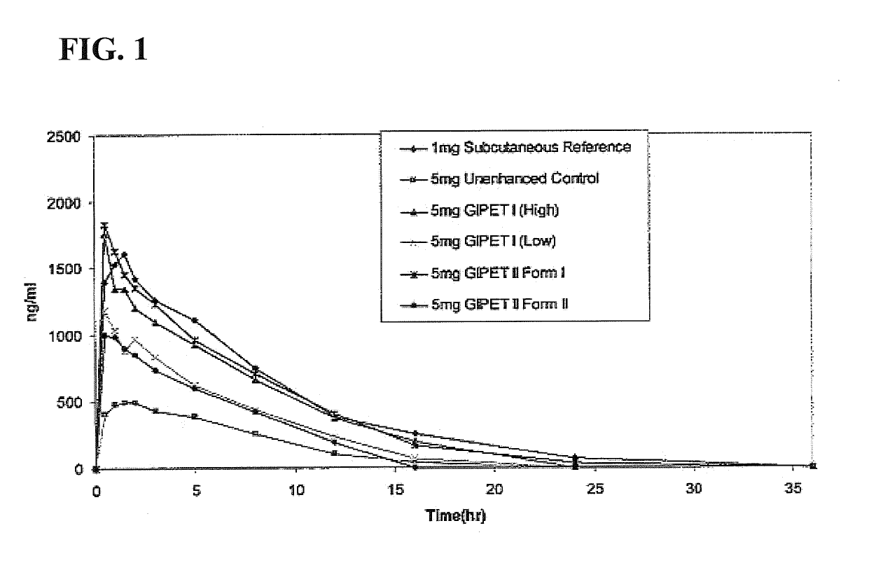

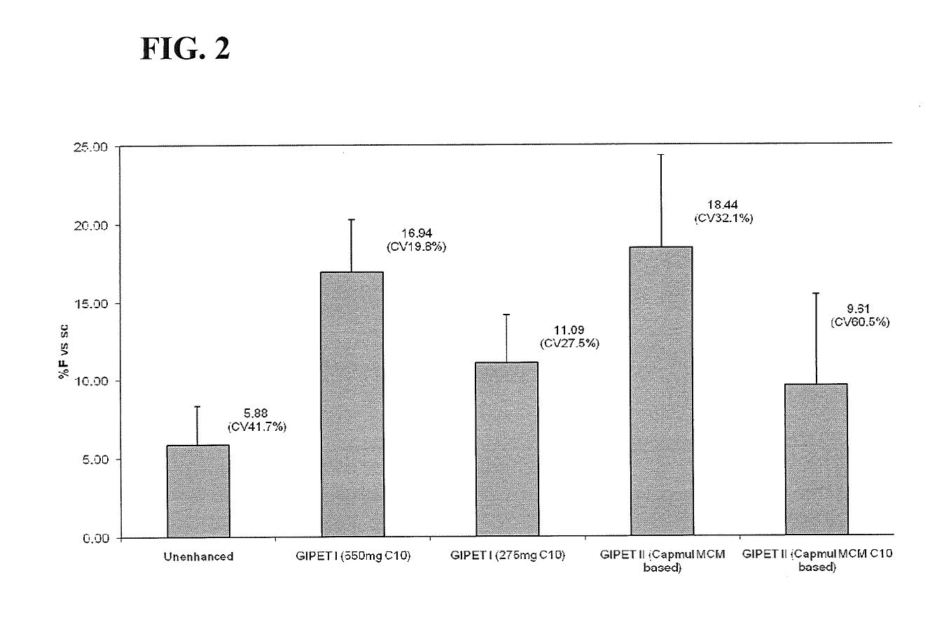

The pharmacokinetic results summary is shown in Table 4 and the raw data is shown in Tables 5-16. Plasma concentration levels are shown in FIG. 1. The 1 mg subcutaneous injection of fondaparinux had a t^{1/2} of 7.3 h (CV 38.5%) and Cₘₐₓ of 1.9 IU/mL. Bioavailability levels and CV are shown in FIG. 2. The bioavailability (Fᵣₑₗ vs. sc) of the unenhanced fondaparinux solution administered to dogs by intra-duodenal instillation was 5.8% (CV 41.7%), with a t^{1/2} of 8.1 h (CV 33.2%) and Cₘₐₓ of 0.6 IU/mL.

Bioavailabilities over 16% of fondaparinux were achieved when administered in either a GIPET™ I or GIPET™ II formulation (Table 4). The bioavailability varied, depending on the formulation type (GIPET™ I or GIPET™ II) and the amount of GIPET I enhancer dosed. Administration of fondaparinux in a GIPET™ I matrix increased the bioavailability up to 16.9% (GIPET I High), while also lowering the variability of absorption (CV 19.8%, GIPET I High). A greater concentration of GIPET™ I enhancer resulted in an increase in absorption of the active. There was a decrease in the variability between animals by increasing the dose of GIPET™ I. Comparison of the Cₘₐₓ showed that variability of the Cₘₐₓ in the GIPET I High formulation (CV 9.0%) was approximately equivalent to the s.c. injection (CV 6.8%).

There was also a significant increase in bioavailability of fondaparinux on administration with GIPET™ II versus the unenhanced formulation (Table 4). Administration of fondaparinux in a GIPET™ II matrix increased the bioavailability up to 18.5% (GIPET™ II Form I) while also lowering the variability of absorption (CV 32.0%, GIPET™ II Form I). The estimates of t^{1/2} were similar after administration of all treatments compared to the subcutaneous injection.

Conclusions from the study are that administration of fondaparinux in a GIPET™ I and/or GIPET™ II formulation significantly increased the bioavailability of the drug and decreased the variability of absorption compared to an unenhanced formulation. Comparable pharmacokinetic profiles were obtained with lead 5 mg fondaparinux/GIPET™ formulations to that shown with the subcutaneous reference injection (1 mg fondaparinux).

TABLE 4
GIPET ™/fondaparinux mean pharmacokinetic parameters (mean ± SD, CV %)

| | PK Parameters | | | | | |
|---|---|---|---|---|---|---|
| | 1 mg fondaparinux | 5 mg fondaparinux | 5 mg fondaparinux + | 5 mg fondaparinux + | 5 mg fondaparinux + | 5 mg fondaparinux + |
| | s.c. | unenhanced | GIPET ™ I (High) | GIPET ™ I (Low) | GIPET ™ II (form I) | GIPET ™ II (form II) |
| Test Item | 1 | 2 | 3 | 4 | 5 | 6 |
| AUC | 14430.54 ± 887.57 | 4248.28 ± 1797.46 | 12124.57 ± 1929.77 | 7951.51 ± 1937.05 | 13170.18 ± 3830.28 | 6915.71 ± 4054.15 |
| (ng/mL · h) | | | | | | |
| CV % | 6.15 | 42.24 | 15.92 | 24.36 | 29.09 | 58.62 |
| Fᵣₑₗ vs. s.c. | — | 5.88 ± 2.45 | 16.94 ± 3.35 | 11.09 ± 3.05 | 18.48 ± 5.91 | 9.61 ± 5.82 |
| (%) | | | | | | |
| CV % | | 41.68 | 19.77 | 27.50 | 32.00 | 60.51 |
| Fᵣₑₗ vs. | — | — | 320.92 ± 118.67 | 211.29 ± 94.42 | 340.18 ± 138.93 | 194.09 ± 152.55 |
| unenhanced | | | | | | |
| (%) | | | | | | |
| CV % | | | 36.98 | 44.69 | 40.84 | 78.60 |
| T^{1/2} (h) | 7.34 ± 2.83 | 8.12 ± 2.70 | 5.77 ± 1.26 | 5.68 ± 1.73 | 6.13 ± 1.30 | 5.33 ± 0.91 |
| CV % | 38.54 | 33.22 | 21.77 | 30.47 | 21.26 | 17.00 |
| Cₘₐₓ | 1654.31 ± 112.21 | 511.90 ± 163.49 | 1761.59 ± 159.17 | 1297.64 ± 260.87 | 1833.96 ± 498.06 | 1027.3 ± 675.93 |
| (ng/mL) | | | | | | |
| CV % | 6.78 | 31.94 | 9.04 | | 27.16 | 65.80 |

TABLE 5
Subcutaneous Reference Leg

| SC LEG | Dog no. 3818 | Dog no. 2640 | Dog no. 7465 | Dog no. 9261 | Dog no. 7107 | | |
|---|---|---|---|---|---|---|---|
| Time hr | ng/ml | ng/ml | ng/ml | ng/ml | ng/ml | Mean ng/ml | Std dev. |
| 0 | 0.00 | 0.00 | 0.00 | 0.00 | 0.00 | 0.00 | 0.00 |
| 0.5 | 1717.97 | 1491.24 | 1491.24 | 1164.21 | 1111.89 | 1395.31 | 253.11 |
| 1 | 1561.00 | 1491.24 | 1438.91 | 1543.56 | 1635.13 | 1533.97 | 74.02 |
| 1.5 | 1517.40 | 1478.15 | 1792.10 | 1635.13 | 1635.13 | 1611.58 | 122.85 |
| 2 | 1334.26 | 1334.26 | 1451.99 | 1399.67 | 1595.88 | 1423.21 | 108.42 |
| 3 | 1151.13 | 1308.10 | 1124.97 | 1447.63 | 1255.78 | 1257.52 | 129.99 |
| 5 | 1138.05 | 1007.24 | 1063.92 | 1164.21 | 1186.01 | 1111.89 | 74.45 |
| 8 | 767.42 | 710.74 | 797.94 | 693.29 | 784.86 | 750.85 | 46.29 |
| 12 | 366.27 | 436.03 | 409.87 | 313.94 | 409.87 | 387.20 | 48.00 |
| 16 | 248.54 | 235.46 | 270.34 | 331.39 | 200.58 | 257.26 | 48.55 |
| 24 | 0.00 | 174.41 | 178.77 | 0.00 | 0.00 | 70.64 | 96.74 |
| 36 | 0.00 | 0.00 | 0.00 | 0.00 | 0.00 | 0.00 | 0.00 |

| | | | | | | MEAN | Std dev. | % RSD |
|---|---|---|---|---|---|---|---|---|
| AUC | 13612.98 | 15099.85 | 15639.44 | 13973.79 | 13826.63 | 14430.54 | 887.57 | 6.15 |
| t1/2 | 4.92 | 9.79 | 10.40 | 7.51 | 4.06 | 7.34 | 2.83 | 38.54 |
| Cmax | 1717.97 | 1491.24 | 1792.10 | 1635.13 | 1635.13 | 1654.31 | 112.21 | 6.78 |

TABLE 6
Subcutaneous Reference Leg Summary

| | AUC | t½ | Cmax |
|---|---|---|---|
| | 15.61 | 4.92 | 1.97 |
| | 17.32 | 9.79 | 1.71 |
| | 17.93 | 10.40 | 2.06 |
| | 16.02 | 7.51 | 1.88 |
| | 15.86 | 4.06 | 1.88 |
| MEAN | 16.55 | 7.34 | 1.90 |
| Std dev. | 1.02 | 2.83 | 0.13 |
| % RSD | 6.15 | 38.54 | 6.78 |

TABLE 8
Test Item 2 Unenhanced Summary

| | | | % F vs | | |
|---|---|---|---|---|---|
| | AUC | SC AUC | sc | t1/2 | Cmax |
| | 2.96 | 15.61 | 3.80 | 7.51 | 0.39 |
| | 7.38 | 17.32 | 8.52 | 11.77 | 0.66 |
| | 3.68 | 17.93 | 4.10 | 9.97 | 0.50 |
| | 3.53 | 16.02 | 4.40 | 5.47 | 0.51 |
| | 6.82 | 15.86 | 8.60 | 5.89 | 0.88 |
| MEAN | 4.87 | 16.55 | 5.88 | 8.12 | 0.59 |
| Std dev. | 2.06 | | 2.45 | 2.70 | 0.19 |
| % RSD | 42.27 | | 41.68 | 33.22 | 31.94 |

TABLE 7
Test Item 2 Unenhanced

| LEG 2 | Dog no. 3818 | Dog no. 2640 | Dog no. 7465 | Dog no. 9261 | Dog no. 7107 | | |
|---|---|---|---|---|---|---|---|
| Time hr | ng/ml | ng/ml | ng/ml | ng/ml | ng/ml | Mean ng/ml | Std dev. |
| 0.00 | 0.00 | 0.00 | 0.00 | 0.00 | 0.00 | 0.00 | 0.00 |
| 0.50 | 218.02 | 370.63 | 318.30 | 353.19 | 763.06 | 404.64 | 208.90 |
| 1.00 | 331.39 | 492.72 | 392.43 | 414.23 | 758.70 | 477.89 | 167.25 |
| 1.50 | 340.11 | 575.56 | 409.87 | 444.75 | 697.65 | 493.59 | 142.58 |
| 2.00 | 340.11 | 549.40 | 436.03 | 401.15 | 723.82 | 490.10 | 151.22 |
| 3.00 | 305.22 | 470.92 | 374.99 | 366.27 | 636.61 | 430.80 | 129.44 |
| 5.00 | 296.50 | 462.20 | 305.22 | 366.27 | 497.08 | 385.45 | 90.92 |
| 8.00 | 196.22 | 270.34 | 283.42 | 200.58 | 348.83 | 259.88 | 63.53 |
| 12.00 | 0.00 | 309.58 | 0.00 | 0.00 | 218.02 | 105.52 | 148.07 |
| 16.00 | 0.00 | 209.30 | 0.00 | 0.00 | 0.00 | 41.86 | 93.60 |
| 24.00 | 0.00 | 0.00 | 0.00 | 0.00 | 0.00 | 0.00 | 0.00 |
| 36.00 | 0.00 | 0.00 | 0.00 | 0.00 | 0.00 | 0.00 | 0.00 |

| | | | | | | MEAN | Std dev. | % RSD |
|---|---|---|---|---|---|---|---|---|
| AUC | 2585.68 | 6433.68 | 3204.85 | 3074.04 | 5943.14 | 4248.28 | 1794.46 | 42.24 |
| SC AUC | 13612.98 | 15099.85 | 15639.44 | 13973.79 | 13826.63 | 14430.54 | | |
| % Bio | 3.80 | 8.52 | 4.10 | 4.40 | 8.60 | 5.88 | 2.45 | 41.68 |
| t1/2 | 7.51 | 11.77 | 9.97 | 5.47 | 5.89 | 8.12 | 2.70 | 33.22 |
| Cmax ng/ml | 340.11 | 575.56 | 436.03 | 444.75 | 763.06 | 511.90 | 163.49 | 31.94 |

TABLE 9
Test Item 3 GIPET I High

| LEG 3 | Dog no. 3818 | Dog no. 2640 | Dog no. 7465 | Dog no. 9261 | Dog no. 7107 | | |
|---|---|---|---|---|---|---|---|
| Time hr | ng/ml | ng/ml | ng/ml | ng/ml | ng/ml | Mean ng/ml | Std dev. |
| 0 | 0 | 0 | 0 | 0 | 0 | 0.00 | 0.00 |
| 0.5 | 1909.83 | 1765.94 | 1508.68 | 1739.78 | 1883.67 | 1761.58 | 159.17 |
| 1 | 1465.07 | 1347.34 | 1059.56 | 1517.40 | 1334.26 | 1344.73 | 177.29 |
| 1.5 | 1229.62 | 1295.02 | 1360.43 | 1543.56 | 1308.10 | 1347.34 | 119.17 |
| 2 | 1399.67 | 1260.14 | 797.94 | 1386.59 | 1177.29 | 1204.33 | 245.14 |
| 3 | 1225.26 | 1050.84 | 837.18 | 1033.40 | 1351.70 | 1099.68 | 196.83 |
| 5 | 898.23 | 898.23 | 688.93 | 1181.65 | 976.72 | 928.75 | 177.23 |
| 8 | 662.77 | 606.09 | 510.16 | 815.38 | 702.01 | 659.28 | 113.22 |
| 12 | 361.91 | 401.15 | 261.62 | 409.87 | 449.11 | 376.73 | 71.42 |
| 16 | 0.00 | 231.10 | 174.41 | 252.90 | 348.83 | 201.45 | 129.00 |
| 24 | 0.00 | 0.00 | 0.00 | 0.00 | 0.00 | 0.00 | 0.00 |
| 36 | 0.00 | 0.00 | 0.00 | 0.00 | 0.00 | 0.00 | 0.00 |

| | | | | | | MEAN | Std dev. | % RSD |
|---|---|---|---|---|---|---|---|---|
| AUC | 11202.80 | 12083.59 | 9419.42 | 13955.26 | 13961.80 | 12124.57 | 1929.77 | 15.92 |
| SC AUC | 13612.98 | 15099.85 | 15639.44 | 13973.79 | 13826.63 | 14430.54 | 887.57 | 6.15 |
| % Bio | 16.46 | 16.00 | 12.05 | 19.97 | 20.20 | 16.94 | 3.35 | 19.77 |
| t1/2 | 5.29 | 5.75 | 5.17 | 4.74 | 7.93 | 5.77 | 1.26 | 21.77 |
| Cmax | 1909.83 | 1765.94 | 1508.68 | 1739.78 | 1883.67 | 1761.58 | 159.17 | 9.04 |

TABLE 10
Test Item 3 GIPET I High Summary

| | | | % F vs | | | Unenhanced | % Frel vs |
|---|---|---|---|---|---|---|---|
| | AUC | SC AUC | sc | t1/2 | Cmax | AUC | unenhanced |
| | 12.85 | 15.61 | 16.46 | 5.29 | 2.19 | 2.96 | 433.99 |
| | 13.86 | 17.32 | 16.00 | 5.75 | 2.03 | 7.38 | 187.82 |
| | 10.80 | 17.93 | 12.04 | 5.17 | 1.73 | 3.68 | 293.88 |
| | 16.00 | 16.02 | 19.98 | 4.74 | 2.00 | 3.53 | 453.97 |
| | 16.01 | 15.86 | 20.20 | 7.93 | 2.16 | 6.82 | 234.92 |
| MEAN | 13.90 | 16.55 | 16.94 | 5.77 | 2.02 | | 320.92 |
| Std dev. | 2.21 | 1.02 | 3.35 | 1.26 | 0.18 | | 118.67 |
| % RSD | 15.92 | 6.15 | 19.79 | 21.77 | 9.04 | | 36.98 |

TABLE 12
Test Item 4 GIPET I Low Summary

| | | | % F vs | | | Unenhanced | % Frel vs |
|---|---|---|---|---|---|---|---|
| | AUC | SC AUC | sc | t1/2 | Cmax | AUC | unenhanced |
| | 10.63 | 15.61 | 13.62 | 3.62 | 1.74 | 2.96 | 359.21 |
| | 8.28 | 17.32 | 9.56 | 6.93 | 1.02 | 7.38 | 112.20 |
| | 8.86 | 17.93 | 9.88 | 5.66 | 1.50 | 3.68 | 240.99 |
| | 6.03 | 16.02 | 7.53 | 7.80 | 1.76 | 3.53 | 171.06 |
| | 11.79 | 15.86 | 14.87 | 4.39 | 1.43 | 6.82 | 173.02 |
| MEAN | 9.12 | | 11.09 | 5.68 | 1.49 | | 211.29 |
| Std dev. | 2.22 | | 3.05 | 1.73 | 0.30 | | 94.42 |
| % RSD | 24.37 | | 27.50 | 30.47 | 20.10 | | 44.69 |

TABLE 11
Test Item 4 GIPET I Low

| LEG 4 | Dog no. 3818 | Dog no. 2640 | Dog no. 7465 | Dog no. 9261 | Dog no. 7107 | | |
|---|---|---|---|---|---|---|---|
| Time hr | ng/ml | ng/ml | ng/ml | ng/ml | ng/ml | Mean ng/ml | Std dev. |
| 0 | 0.00 | 0.00 | 0.00 | 0.00 | 0.00 | 0.00 | 0.00 |
| 0.5 | 1517.40 | 863.35 | 1308.10 | 1530.48 | 680.21 | 1179.91 | 388.31 |
| 1 | 1312.46 | 889.51 | 1207.81 | 562.48 | 1177.29 | 1029.91 | 304.72 |
| 1.5 | 1229.62 | 667.13 | 915.67 | 575.56 | 981.08 | 873.81 | 260.54 |
| 2 | 1229.62 | 723.82 | 1068.28 | 575.56 | 1242.70 | 968.00 | 303.15 |
| 3 | 1098.81 | 601.73 | 854.63 | 466.56 | 1133.69 | 831.08 | 295.50 |
| 5 | 806.66 | 514.52 | 597.37 | 392.43 | 802.30 | 622.66 | 181.29 |
| 8 | 514.52 | 388.07 | 405.51 | 279.06 | 601.73 | 437.78 | 123.97 |
| 12 | 213.66 | 218.02 | 252.90 | 209.30 | 274.70 | 233.71 | 28.69 |
| 16 | 0.00 | 174.41 | 0.00 | 0.00 | 170.05 | 68.89 | 94.35 |
| 24 | 0.00 | 0.00 | 0.00 | 0.00 | 0.00 | 0.00 | 0.00 |
| 36 | 0.00 | 0.00 | 0.00 | 0.00 | 0.00 | 0.00 | 0.00 |

| | | | | | | MEAN | Std dev. | % RSD |
|---|---|---|---|---|---|---|---|---|
| AUC | 9272.26 | 7218.54 | 7723.25 | 5260.75 | 10282.77 | 7951.51 | 1937.05 | 24.36 |
| SC AUC | 13612.98 | 15099.85 | 15639.44 | 13973.79 | 13826.63 | | | |
| % Bio | 13.62 | 9.56 | 9.88 | 7.53 | 14.87 | 11.09 | 3.05 | 27.50 |
| t1/2 | 3.62 | 6.93 | 5.66 | 7.80 | 4.39 | 5.68 | 1.73 | 30.47 |
| Cmax | 1517.40 | 889.51 | 1308.10 | 1530.48 | 1242.70 | 1297.64 | 260.87 | 20.10 |

TABLE 13
Test Item 5 GIPET II

| | LEG 5 | | | | | | | |
|---|---|---|---|---|---|---|---|---|
| | Dog no. 3818 | Dog no. 2640 | Dog no. 7465 | Dog no. 9261 | Dog no. 7107 | Mean | | |
| Time hr | ng/ml | ng/ml | ng/ml | ng/ml | ng/ml | ng/ml | Std dev. | |
| 0 | 0.00 | 0.00 | 0.00 | 0.00 | 0.00 | 0.00 | 0.00 | |
| 0.5 | 2158.37 | 1792.10 | 994.16 | 2223.77 | 2001.40 | 1833.96 | 498.06 | |
| 1 | 1752.86 | 1700.53 | 837.18 | 2066.80 | 1792.10 | 1629.89 | 465.21 | |
| 1.5 | 1504.32 | 1543.56 | 906.95 | 1792.10 | 1517.40 | 1452.86 | 327.16 | |
| 2 | 1491.24 | 1229.62 | 872.07 | 1622.05 | 1556.64 | 1354.32 | 308.01 | |
| 3 | 1273.22 | 1247.06 | 758.70 | 1465.07 | 1399.67 | 1228.74 | 277.63 | |
| 5 | 946.19 | 1098.81 | 558.12 | 1103.17 | 1098.81 | 961.02 | 234.90 | |
| 8 | 758.70 | 715.10 | 414.23 | 854.63 | 780.50 | 704.63 | 170.01 | |
| 12 | 348.83 | 392.43 | 279.06 | 449.11 | 553.76 | 404.64 | 103.98 | |
| 16 | 0.00 | 313.94 | 0.00 | 261.62 | 279.06 | 170.93 | 157.17 | |
| 24 | 0.00 | 0.00 | 0.00 | 0.00 | 170.05 | 34.01 | 76.05 | |
| 36 | 0.00 | 0.00 | 0.00 | 0.00 | 0.00 | 0.00 | 0.00 | |
| | | | | | | MEAN | Std dev. | % RSD |
| AUC | 12152.26 | 14014.13 | 7122.61 | 15570.77 | 16991.15 | 13170.18 | 3830.28 | 29.08 |
| SC AUC | 13612.98 | 15099.85 | 15639.44 | 13973.79 | 13826.63 | | | |
| % Bio | 17.85 | 18.56 | 9.11 | 22.29 | 24.58 | 18.48 | 5.91 | 32.00 |
| t½ | 4.77 | 6.74 | 7.00 | 4.68 | 7.44 | 6.13 | 1.30 | 21.26 |
| Cmax ng/ml | 2158.37 | 1792.10 | 994.16 | 2223.77 | 2001.40 | 1833.96 | 498.06 | 27.16 |

TABLE 14
Test Item 5 GIPET II Summary

| | | | % F | | | | |
|---|---|---|---|---|---|---|---|
| | | SC | vs | | | Unenhanced | % Frel vs |
| | AUC | AUC | sc | t½ | Cmax | AUC | unenhanced |
| | 13.94 | 15.61 | 17.85 | 4.77 | 2.48 | 2.96 | 470.78 |
| | 15.89 | 17.32 | 18.35 | 6.74 | 2.06 | 7.38 | 215.38 |
| | 8.17 | 17.93 | 9.11 | 7.00 | 1.14 | 3.68 | 222.24 |
| | 17.86 | 16.02 | 22.29 | 4.68 | 2.55 | 3.53 | 506.67 |
| | 19.48 | 15.86 | 24.58 | 7.44 | 2.30 | 6.82 | 285.84 |
| MEAN | 15.07 | | 18.44 | 6.13 | 2.10 | | 340.18 |
| Std dev. | 4.38 | | 5.92 | 1.30 | 0.57 | | 138.93 |
| % RSD | 29.09 | | 32.08 | 21.26 | 27.16 | | 40.84 |

TABLE 16
Test Item 6 GIPET II Summary

| | | | % F | | | | |
|---|---|---|---|---|---|---|---|
| | | SC | vs | | | Unenhanced | % Frel vs |
| | AUC | AUC | sc | t½ | Cmax | AUC | unenhanced |
| | 3.76 | 15.61 | 4.82 | 6.22 | 0.50 | 2.96 | 127.11 |
| | 5.13 | 17.32 | 5.92 | 5.97 | 0.67 | 7.38 | 69.47 |
| | 8.70 | 17.93 | 9.70 | 4.24 | 1.26 | 3.68 | 236.77 |
| | 15.58 | 16.02 | 19.45 | 4.48 | 2.46 | 3.53 | 441.99 |
| | 6.48 | 15.86 | 8.18 | 5.73 | 1.01 | 6.82 | 95.12 |
| MEAN | 7.93 | | 9.61 | 5.33 | 1.18 | | 194.09 |
| Std dev. | 4.65 | | 5.82 | 0.91 | 0.78 | | 152.55 |
| % RSD | 58.62 | | 60.51 | 17.00 | 65.80 | | 78.60 |

TABLE 15
Test Item 6 GIPET II

| | LEG 6 | | | | | | | |
|---|---|---|---|---|---|---|---|---|
| | Dog no. 3818 | Dog no. 2640 | Dog no. 7465 | Dog no. 9261 | Dog no. 7107 | | | |
| Time hr | ng/ml | ng/ml | ng/ml | ng/ml | ng/ml | Mean ng/ml | Std dev. | |
| 0 | 0.00 | 0.00 | 0.00 | 0.00 | 0.00 | 0.00 | 0.00 | |
| 0.5 | 431.67 | 579.93 | 1098.81 | 2001.40 | 876.43 | 997.65 | 617.91 | |
| 1 | 366.27 | 536.32 | 1090.08 | 2145.29 | 771.78 | 981.95 | 704.78 | |
| 1.5 | 436.03 | 497.08 | 972.36 | 1818.26 | 758.70 | 896.49 | 558.11 | |
| 2 | 436.03 | 340.11 | 928.75 | 1883.67 | 645.33 | 846.78 | 622.07 | |
| 3 | 405.51 | 427.31 | 841.55 | 1438.91 | 558.12 | 734.28 | 430.51 | |
| 5 | 361.91 | 388.07 | 614.81 | 1159.85 | 466.56 | 598.24 | 329.03 | |
| 8 | 235.46 | 313.94 | 488.36 | 732.54 | 331.39 | 420.34 | 197.17 | |
| 12 | 0.00 | 174.41 | 200.58 | 392.43 | 200.58 | 193.60 | 139.18 | |
| 16 | 0.00 | 0.00 | 0.00 | 0.00 | 0.00 | 0.00 | 0.00 | |
| 24 | 0.00 | 0.00 | 0.00 | 0.00 | 0.00 | 0.00 | 0.00 | |
| 36 | 0.00 | 0.00 | 0.00 | 0.00 | 0.00 | 0.00 | 0.00 | |
| | | | | | | MEAN | Std dev. | % RSD |
| AUC | 3281.15 | 4469.35 | 7588.08 | 13586.81 | 5653.18 | 6915.71 | 4054.15 | 58.62 |
| SC AUC | 13612.98 | 15099.85 | 15639.44 | 13973.79 | 13826.63 | | | |
| % Bio | 4.82 | 5.92 | 9.70 | 19.45 | 8.18 | 9.61 | 5.82 | 60.51 |
| t½ | 6.22 | 5.97 | 4.24 | 4.48 | 5.73 | 5.33 | 0.91 | 17.00 |
| Cmax | 436.03 | 579.93 | 1098.81 | 2145.29 | 876.43 | 1027.30 | 675.93 | 65.80 |

### Example 2

### Bioavailability of Enteric-Coated Oral Tablets of Fondaparinux

A pre-clinical study was undertaken to evaluate oral bioavailability of two different doses of Fondaparinux GIPET® tablets (Test Items 2 and 3) versus a subcutaneous (SC) control formulation of drug alone (Test Item 1) in fasted beagle dogs. Evaluation was based on the pharmacokinetic profile of GIPET® based enteric-coated fondaparinux tablets relative to the SC control formulation. Each Test Item was dosed to a group of six male beagle dogs with a washout period of 7 days between each dose. The test items evaluated and their route of administration are shown in Table 17. Following dose administration, blood samples were collected at scheduled intervals (see below) and analyzed for fondaparinux plasma concentrations. The fondaparinux concentrations were determined using an anti-factor Xa based diagnostic kit. The performance of oral tablet formulations was evaluated against a subcutaneous control formulation (Test Item I) to assess oral bioavailability. Plasma drug concentration versus time profiles were used for estimating 1) pharmacokinetic parameters, and 2) oral bioavailability.

TABLE 17
Test Items and fondaparinux formulations dosed

| Test | Test Item | | | Amount |
|---|---|---|---|---|
| Item | Name | Formulation | Route | API |
| 1 | Aqueous | 5 mg/mL | Subcutaneous | 0.5 mL of |
| | formulation of | Fondaparinux | | test item |
| | Fondaparinux | Sterile Aqueous | | 2.5 mg/ |
| | | Formulation | | animal |
| 2 | GIPET ® | GIPET ® I tablets | Oral | 1 tablet |
| | I Tablet I | containing | | 12.5 mg/ |
| | | 12.5 mg | | animal |
| | | Fondaparinux | | |
| 3 | GIPET ® | GIPET ® I tablets | Oral | 1 tablet |
| | I Tablet II | containing 20 mg | | 20 mg/ |
| | | Fondaparinux | | animal |

Test Item 1 was administered subcutaneously and test Items 2 and 3 were administered orally as single tablet doses. Blood samples for Test Item 1 (control formulation) were collected and analyzed for plasma fondaparinux levels at pre-dose (T0), 10, 20, 40 minutes, 1, 1.5, 2, 3, 4, 6, 10, 14 and 24 hours following test article administration. Blood samples for Test Items 2 and 3 (GIPET® tablets) were collected and analyzed for plasma fondaparinux levels at pre-dose (T0), 15, 30 minutes, 1, 1.5, 2, 3, 4, 5, 7, 10, 14 and 24 hours following test article administration. Upon receipt of bio-analytical data, the individual animal plasma concentration data was loaded into an Excel spreadsheet (Microsoft® office Excel 2003). Pharmacokinetic parameters including AUC₀₋ₜ, Cₘₐₓ, Tₘₐₓ and relative oral bioavailability were calculated using macros written for MS Excel by Uransky et al., PK functions for Microsoft Excel, available at www.boomer.org/pkin/xcel/pkf.pkf.doc.

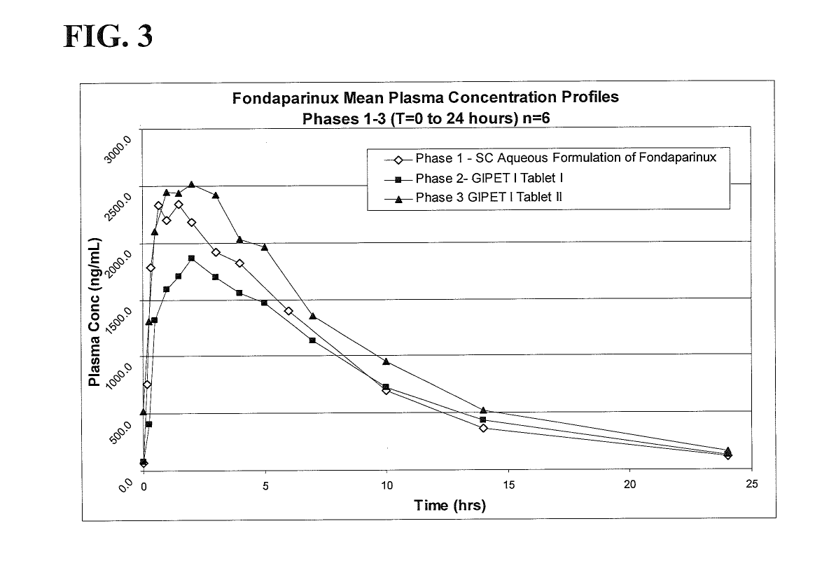

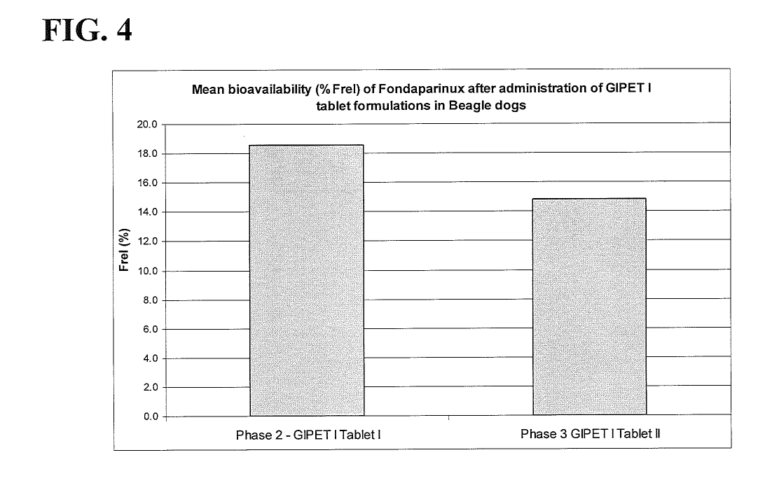

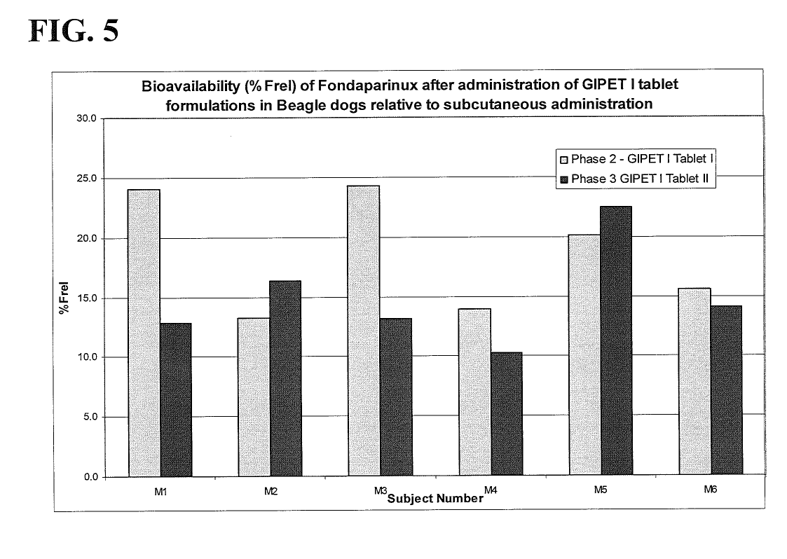

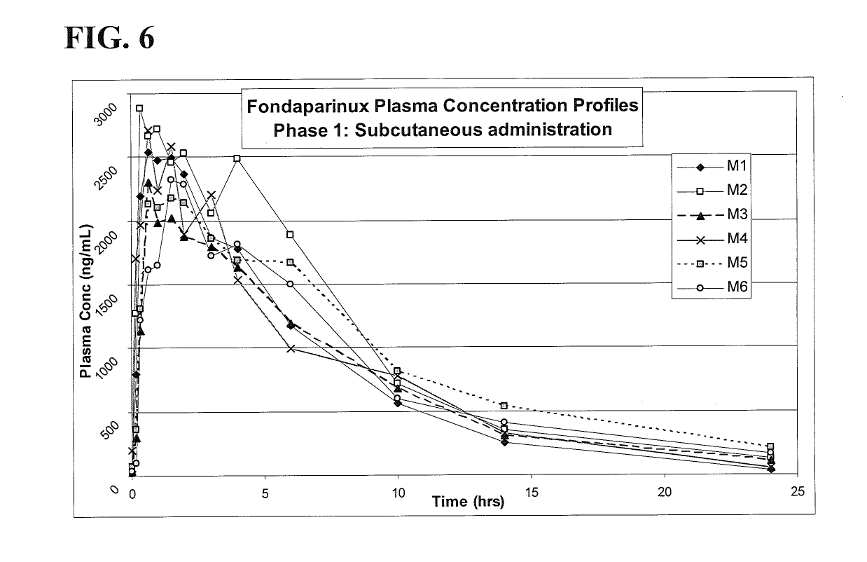

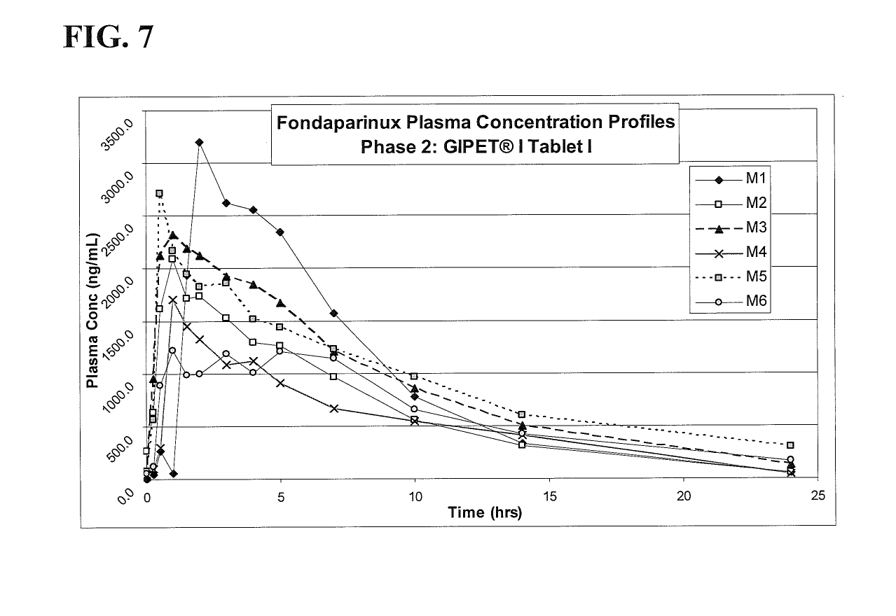

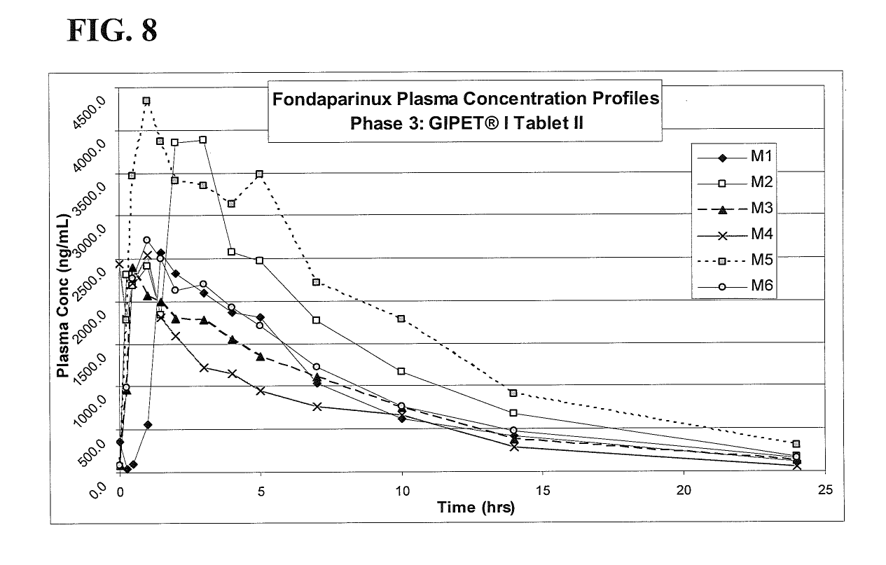

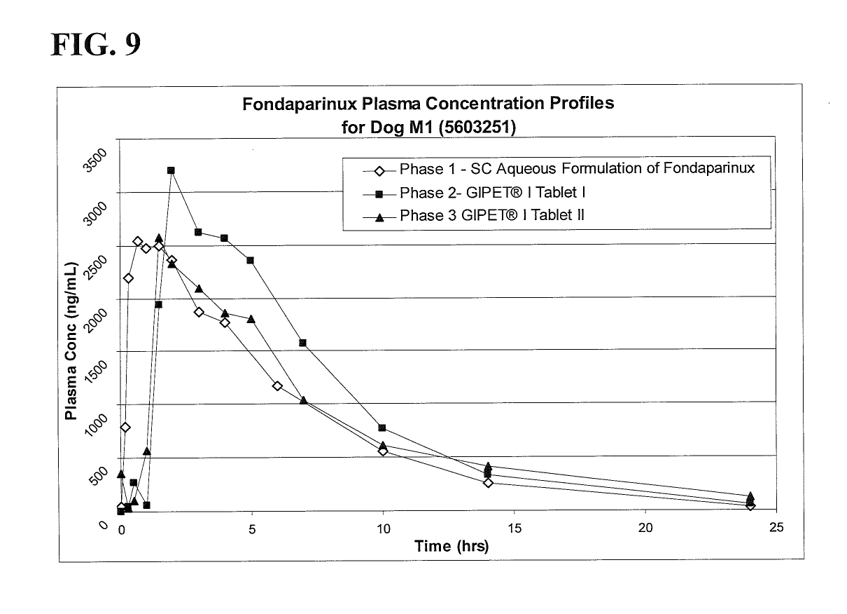

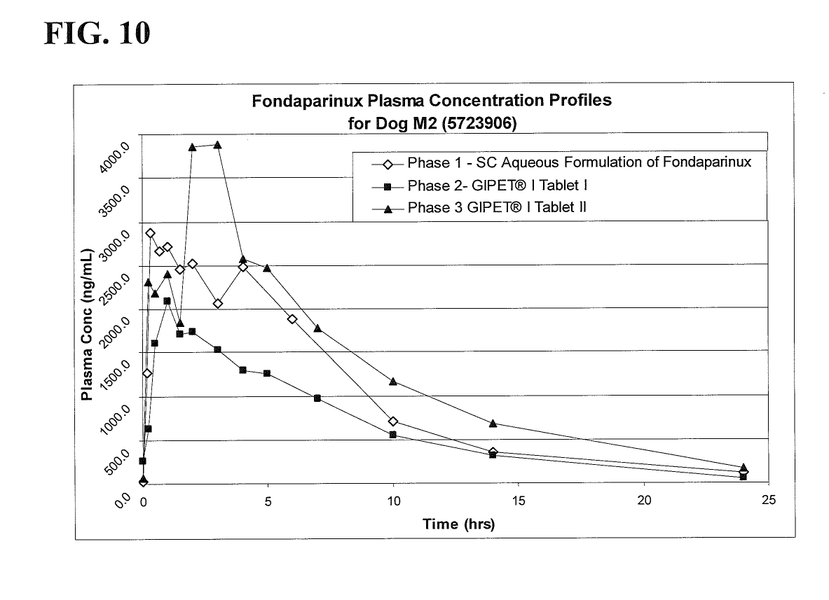

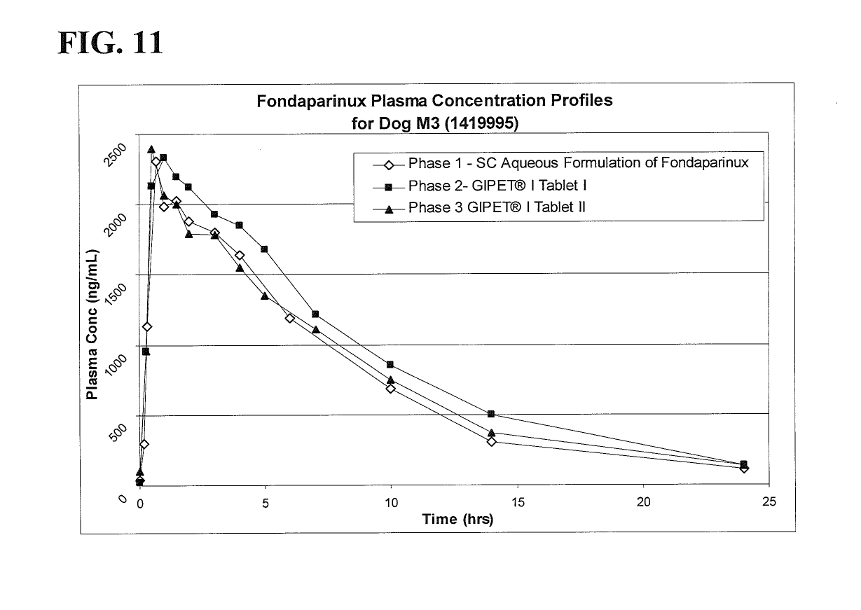

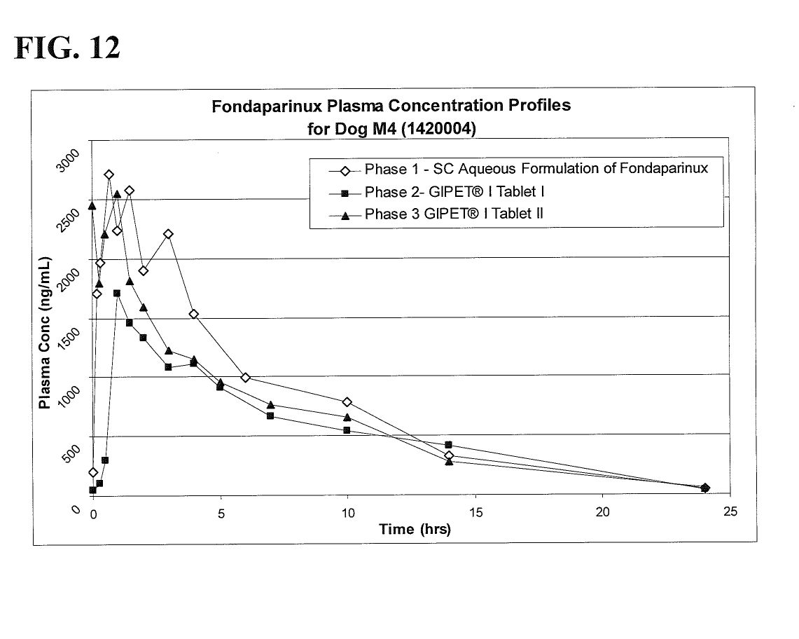

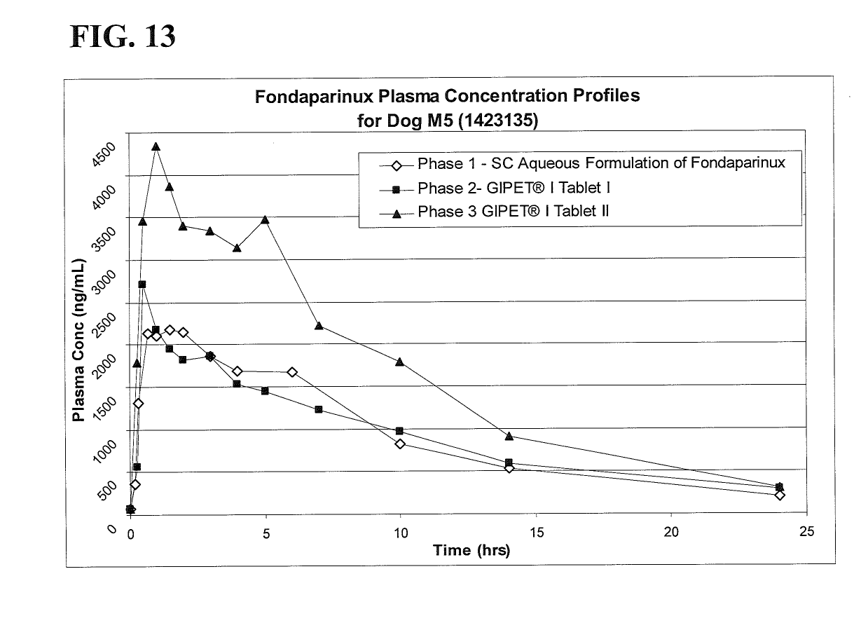

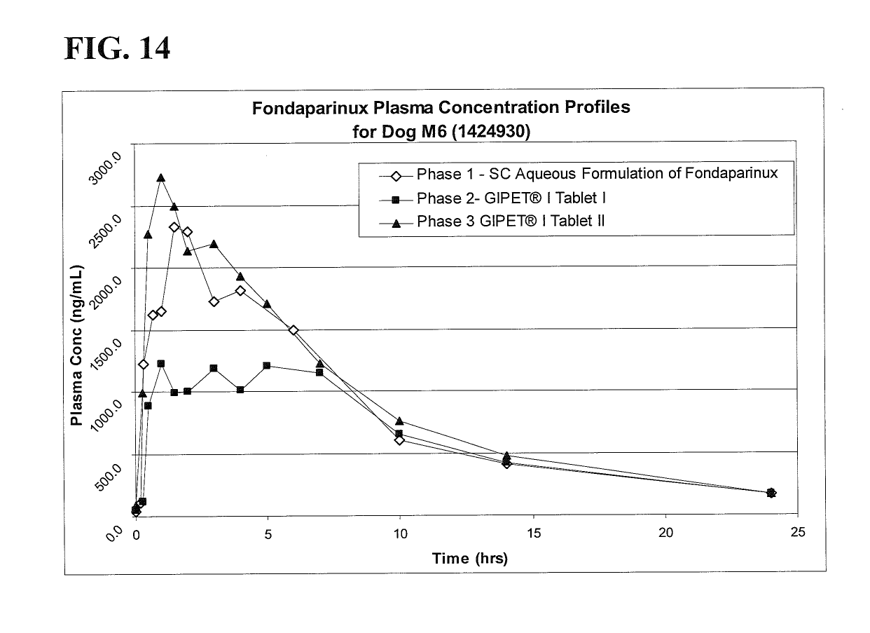

A summary of the pharmacokinetic parameters is presented in Table 18. The systemic absorption of fondaparinux from the various test items and comparisons between these test items are illustrated in FIGS. 3 to 8. The systemic absorption from various test items is shown in FIGS. 4 and 5. The data were re-plotted in FIGS. 9 to 14 to depict comparative absorption in each dog. Plasma concentration data for each dog is detailed in Tables 19 to 21.

TABLE 18
Mean Pharmacokinetic Parameters, (Mean ± SD, (CV %)); n = 6

| | Reference | | |
|---|---|---|---|
| | Subcutaneous | GIPET ® I | GIPET ® I |
| PK Parameters | Dose | Tablet I | Tablet II |
| AUC₀₋ₜ (ng/ml · hr) | 19826 ± 2423 | 18103 ± 4040 | 24068 ± 9361 |
| (% CV) | (12) | (22) | (39) |
| Cₘₐₓ (ng/ml) | 2489 ± 269 | 2210 ± 706 | 3077 ± 824 |
| (% CV) | (11) | (32) | (27) |
| Tₘₐₓ (hours) | 0.9 ± 0.5 | 0.8 ± 0.4 | 0.9 ± 0.6 |
| T_{1/2} (hours) | 5.5 ± 1.7 | 5.4 ± 2 | 5.5 ± 0.8 |
| (10-24 hour | | | |
| interval) | | | |
| Fᵣₑₗ vs unenhanced | N/A | 18.5 ± 5 | 14.9 ± 4 |
| (% CV) | | (27) | (28) |

TABLE 19
Plasma drug concentration and PK data of individual dogs after subcutaneous administration of Test Item I

| | M1 | M2 | M3 | M4 | M5 | M6 | | | |
|---|---|---|---|---|---|---|---|---|---|
| TIME | (5603251) | (5723906) | (1419995) | (1420004) | (1423135) | (1424930) | Mean | SD | % RSD |
| 0 | 40.6* | 20.0* | 40.2* | 199 | 74.3* | 36.0* | 68.4 | 66.4 | 97.2 |
| 10 min | 796 | 1274 | 297 | 1712 | 362 | 102* | 757.2 | 628.9 | 83.1 |
| 20 min | 2198 | 2880 | 1135 | 1966 | 1306 | 1220 | 1784.2 | 689.0 | 38.6 |
| 40 min | 2542 | 2668 | 2304 | 2708 | 2132 | 1620 | 2329.0 | 411.4 | 17.7 |
| 1 h | 2478 | 2716 | 1984 | 2242 | 2106 | 1650 | 2196.0 | 375.0 | 17.1 |
| 1.5 h | 2494 | 2460 | 2022 | 2580 | 2176 | 2326 | 2343.0 | 211.6 | 9.0 |
| 2 h | 2368 | 2526 | 1880 | 1900 | 2142 | 2288 | 2184.0 | 259.4 | 11.9 |
| 3 h | 1874 | 2062 | 1798 | 2206 | 1864 | 1726 | 1921.7 | 178.7 | 9.3 |
| 4 h | 1776 | 2484 | 1638 | 1532 | 1690 | 1814 | 1822.3 | 339.4 | 18.6 |
| 6 h | 1171 | 1888 | 1192 | 996 | 1670 | 1498 | 1402.5 | 340.5 | 24.3 |
| 10 h | 558 | 712 | 682 | 780 | 816 | 598 | 691.0 | 100.4 | 14.5 |
| 14 h | 253 | 354 | 307 | 329 | 533 | 404 | 363.3 | 97.0 | 26.7 |
| 24 h | 36.0* | 123* | 110* | 51.1* | 208 | 165* | 115.5 | 65.7 | 56.8 |
| AUC₍₀₋ₜ₎ | 17822.3 | 23472.3 | 17610.3 | 18515.6 | 22119.6 | 19417.5 | 19826.3 | 2423.4 | 12.2 |
| (ng/mL * hr) | | | | | | | | | |
| T_{1/2} | 3.5 | 5.7 | 5.5 | 3.6 | 7.1 | 7.6 | 5.5 | 1.7 | 30.9 |
| Cₘₐₓ | 2542.0 | 2880.0 | 2304.0 | 2708.0 | 2176.0 | 2326.0 | 2489.3 | 269.2 | 10.8 |
| (ng/mL) | | | | | | | | | |
| Tₘₐₓ | 0.7 | 0.3 | 0.7 | 0.7 | 1.5 | 1.5 | 0.9 | 0.5 | 55.2 |
| (hrs) | | | | | | | | | |
| Fᵣₑₗ (vs | N/A | N/A | N/A | N/A | N/A | N/A | N/A | N/A | N/A |
| subcutaneous) | | | | | | | | | |

*Below LLOQ of method

TABLE 20
Plasma drug concentration and PK data of individual dogs after subcutaneous administration of Test Item II

| | M1 | M2 | M3 | M4 | M5 | M6 | | | |
|---|---|---|---|---|---|---|---|---|---|
| TIME (hr) | (5603251) | (5723906) | (1419995) | (1420004) | (1423135) | (1424930) | Mean | SD | % RSD |
| 0 | 2.0* | 267.0 | 23.3* | 43.6* | 75.5* | 51.2* | 77.1 | 96.3 | 124.9 |
| 15 min | 48.0* | 631 | 954 | 104* | 566 | 118* | 403.5 | 368.5 | 91.3 |
| 30 min | 267 | 1612 | 2130 | 295 | 2716 | 886 | 1317.7 | 1003.8 | 76.2 |
| 1 h | 53.4* | 2096 | 2330 | 1704 | 2172 | 1220 | 1595.9 | 854.8 | 53.6 |
| 1.5 h | 1934 | 1714 | 2194 | 1452 | 1944 | 988 | 1704.3 | 430.5 | 25.3 |
| 2 h | 3196 | 1734 | 2124 | 1332 | 1822 | 1002 | 1868.3 | 759.8 | 40.7 |
| 3 h | 2620 | 1532 | 1928 | 1082 | 1864 | 1184 | 1701.7 | 566.0 | 33.3 |
| 4 h | 2560 | 1296 | 1852 | 1114 | 1522 | 1006 | 1558.3 | 576.6 | 37.0 |
| 5 h | 2350 | 1258 | 1678 | 904 | 1442 | 1202 | 1472.3 | 501.1 | 34.0 |
| 7 h | 1568 | 968 | 1210 | 666 | 1230 | 1146 | 1131.3 | 300.0 | 26.5 |
| 10 h | 774 | 551 | 850 | 544 | 962 | 648 | 721.5 | 169.2 | 23.4 |
| 14 h | 336 | 308 | 501 | 412 | 596 | 422 | 429.2 | 106.5 | 24.8 |
| 24 h | 52.9* | 55.5* | 138* | 39.9* | 295 | 163* | 124.1 | 97.8 | 78.8 |
| AUC₍₀₋ₜ₎ | 21453.6 | 15498.1 | 21389.2 | 12923.6 | 22231.9 | 15118.7 | 18102.5 | 4039.6 | 22.3 |
| (ng/mL * hr) | | | | | | | | | |
| T_{1/2} | 3.6 | 4.2 | 5.3 | 3.5 | 8.5 | 7.1 | 5.4 | 2.0 | 37.5 |
| Cₘₐₓ | 3196.0 | 2096.0 | 2330.0 | 1704.0 | 2716.0 | 1220.0 | 2210.3 | 705.9 | 31.9 |
| (ng/mL) | | | | | | | | | |
| Tₘₐₓ | 1.5 | 0.7 | 0.7 | 0.7 | 0.3 | 0.7 | 0.8 | 0.4 | 52.1 |
| (hrs) | | | | | | | | | |
| Fᵣₑₗ (vs | 24.1 | 13.2 | 24.3 | 14.0 | 20.1 | 15.6 | 18.5 | 5.0 | 26.9 |
| subcutaneous) | | | | | | | | | |

*Below LLOQ of method

TABLE 21
Plasma drug concentration and PK data of individual dogs after subcutaneous administration of Test Item III

| | M1 | M2 | M3 | M4 | M5 | M6 | | | |
|---|---|---|---|---|---|---|---|---|---|
| TIME (hr) | (5603251) | (5723906) | (1419995) | (1420004) | (1423135) | (1424930) | Mean | SD | % RSD |
| 0 | 360.0 | 71.4* | 101* | 2448 | 70.1* | 84.2* | 522.5 | 949.9 | 181.8 |
| 15 min | 36.0* | 2318 | 958 | 1798 | 1788 | 989 | 1314.5 | 816.4 | 62.1 |
| 30 min | 102* | 2186 | 2396 | 2210 | 3460 | 2270 | 2104.0 | 1093.4 | 52.0 |
| 1 h | 565 | 2410 | 2064 | 2544 | 4348 | 2724 | 2442.5 | 1215.7 | 49.8 |
| 1.5 h | 2570 | 1845 | 2000 | 1816 | 3868 | 2498 | 2432.8 | 774.1 | 31.8 |
| 2 h | 2332 | 3855 | 1796 | 1592 | 3404 | 2134 | 2518.8 | 909.2 | 36.1 |
| 3 h | 2098 | 3882 | 1782 | 1224 | 3352 | 2194 | 2422.0 | 999.8 | 41.3 |
| 4 h | 1866 | 2570 | 1554 | 1144 | 3140 | 1930 | 2034.0 | 717.2 | 35.3 |
| 5 h | 1806 | 2476 | 1348 | 952 | 3476 | 1704 | 1960.3 | 899.4 | 45.9 |
| 7 h | 1032 | 1774 | 1112 | 758 | 2216 | 1228 | 1353.3 | 539.0 | 39.8 |
| 10 h | 613 | 1162 | 745 | 656 | 1786 | 759 | 953.5 | 452.2 | 47.4 |
| 14 h | 410 | 679 | 368 | 284 | 910 | 477 | 521.3 | 232.4 | 44.6 |
| 24 h | 119.0* | 168* | 135* | 61.9* | 303 | 163* | 158.3 | 80.5 | 50.9 |
| AUC₍₀₋ₜ₎ | 18272.3 | 30687.9 | 18526.1 | 15242.8 | 39796.3 | 21881.0 | 24067.7 | 9361.3 | 38.9 |
| (ng/mL * hr) | | | | | | | | | |
| T_{1/2} | 5.9 | 5.0 | 5.9 | 4.2 | 5.6 | 6.3 | 5.5 | 0.8 | 14.0 |
| Cₘₐₓ | 2570.0 | 3882.0 | 2396.0 | 2544.0 | 4348.0 | 2724.0 | 3077.3 | 823.8 | 26.8 |
| (ng/mL) | | | | | | | | | |
| Tₘₐₓ | 1.0 | 2.0 | 0.3 | 0.7 | 0.7 | 0.7 | 0.9 | 0.6 | 65.7 |
| (hrs) | | | | | | | | | |
| Fᵣₑₗ (vs | 12.8 | 16.3 | 13.2 | 10.3 | 22.5 | 14.1 | 14.9 | 4.2 | 28.4 |
| subcutaneous) | | | | | | | | | |

*Below LLOQ of method

### Example 3

### Dissolution Studies of Oral Tablets of Fondaparinux

The tablets described in Example 2 were subjected to dissolution studies to study the release of both fondaparinux and sodium caprate from the tablets. The dissolution assays were carried out on enteric-coated tablets under the conditions described in Table 1. The results of the dissolution study for the tablet of Test Item 2 (12.5 mg fondaparinux) are shown in Tables 22 (fondaparinux) and 23 (sodium caprate).

TABLE 22
Dissolution of fondaparinux from 12.5 mg fondaparinux tablet
% Dissolution Results

| Minutes | Acid | 5 | 10 | 20 | 25 | 30 | 35 | 45 | 60 |
|---|---|---|---|---|---|---|---|---|---|
| V1 | ND | ND | ND | 26.2 | 43.4 | 60.7 | 83.0 | 101.0 | 107.4 |
| V2 | ND | ND | ND | 31.3 | 51.2 | 64.4 | 85.1 | 101.8 | 102.7 |
| V3 | ND | ND | ND | 29.2 | 43.7 | 60.8 | 67.9 | 94.2 | 100.3 |
| V4 | ND | ND | ND | 33.8 | 53.9 | 75.2 | 88.8 | 99.9 | 103.5 |
| V5 | ND | ND | ND | 32.5 | 59.0 | 78.0 | 92.6 | 105.1 | 107.2 |
| V6 | ND | ND | ND | 29.9 | 46.8 | 65.5 | 77.2 | 95.7 | 110.6 |
| Mean: | N/A | N/A | N/A | 30.5 | 49.7 | 67.4 | 82.4 | 99.6 | 105.3 |
| % RSD: | N/A | N/A | N/A | 8.8 | 12.4 | 11.0 | 10.7 | 4.1 | 3.6 |

ND = Not Detected
N/A = Not Applicable

TABLE 23
Dissolution of sodium caprate from 12.5 mg fondaparinux tablet
% Dissolution Results

| Minutes | Acid | 5 | 10 | 20 | 25 | 30 | 35 | 45 | 60 |
|---|---|---|---|---|---|---|---|---|---|
| V1 | ND | ND | 1.8 | 22.4 | 40.2 | 57.4 | 72.0 | 91.7 | 98.3 |
| V2 | ND | ND | 4.6 | 31.5 | 53.5 | 70.2 | 83.2 | 92.3 | 100.7 |
| V3 | ND | ND | 5.2 | 30.7 | 47.9 | 63.9 | 77.3 | 95.6 | 100.6 |
| V4 | ND | ND | 4.6 | 32.1 | 53.7 | 71.1 | 85.3 | 96.3 | 98.8 |
| V5 | ND | ND | 3.7 | 31.5 | 54.1 | 70.7 | 84.7 | 97.1 | 98.0 |
| V6 | ND | ND | 4.3 | 29.2 | 47.4 | 62.5 | 74.3 | 91.4 | 97.5 |
| Mean: | N/A | N/A | 4.0 | 29.6 | 49.5 | 66.0 | 79.5 | 95.1 | 99.0 |
| % RSD: | N/A | N/A | 29.9 | 12.3 | 11.0 | 8.5 | 7.2 | 3.0 | 1.4 |

ND = Not Detected
N/A = Not Applicable

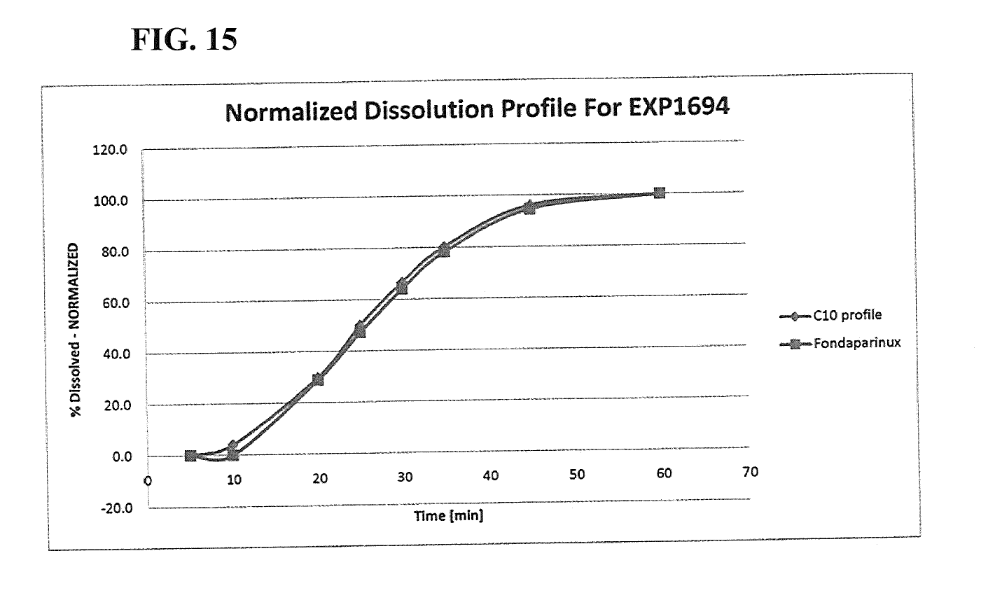

The dissolution data were normalized and the normalized results are shown in Table 24 and plotted in FIG. 15.

TABLE 24
Normalized dissolution data for 12.5 mg fondaparinux tablet

| Time | % Disso C10 | % Disso Fondaparinux |
|---|---|---|
| 5 | 0.0 | 0.0 |
| 10 | 4.0 | 0.0 |
| 20 | 29.9 | 29.0 |
| 25 | 50.0 | 47.2 |
| 30 | 66.7 | 64.0 |
| 35 | 80.3 | 78.3 |
| 45 | 96.1 | 94.6 |
| 60 | 100.0 | 100.0 |

The dissolution data was used to calculate the difference (f1) and similarity (f2) factors. The results are shown in Tables 25 (non-normalized data) and 26 (normalized data). Using both the normalized and non-normalized data, the dissolution rate of sodium caprate and fondaparinux were calculated to be highly similar.

TABLE 25
F1 and F2 calculation using non-normalized data for 12.5 mg fondaparinux
tablet

| F1 calculation | | | | | | |
|---|---|---|---|---|---|---|
| | C10 | | | | | |
| time | (reference) | Fondaparinux | | | | |
| [t] | pre-change | post-change | | | F2 calculation | |
| min | [Rt] | [T] | R − T | ABS [R − T] | | (R − T)² |
| 5 | 0.0 | 0.0 | 0.0 | 0 | | 0.0 |
| 10 | 4.0 | 0.0 | 4.0 | 4 | | 16.00 |
| 20 | 29.6 | 30.5 | −0.9 | 0.9 | | 0.81 |
| 25 | 49.5 | 49.7 | −0.2 | 0.2 | | 0.04 |
| 30 | 66.0 | 67.4 | −1.4 | 1.4 | | 1.96 |
| 35 | 79.5 | 82.4 | −2.9 | 2.9 | | 8.41 |
| 45 | 95.1 | 99.6 | −4.5 | 4.5 | n (timepoints) = 7 | 20.25 |
| *60 | 99.1 | 105.3 | | | | |
| sum | 323.7 | | | | SUM(R − T)² | 47.47 |
| | | | Sum ABS [R − T] | 13.9 | (1/n) | 0.142857143 |
| | | | f1 Factor | 4.29409947 | (1/n) * SUM(R − T)² | 6.781428571 |

| n (timepoints) = 7.0 | | | 1 + [1/n) * SUM(R − T)² | 7.781428571 |
|---|---|---|---|---|
| | | | SQRT | 2.789521208 |
| | | | (1/SQRT) * 100 | 35.84844586 |
| | | | F2 FACTOR | 77.72351662 |
| USP Guidance: Not different if (f1) in range from 0-15 | | | USP Guidance: Similar if (f2) in range | |
| | | | of 50-100 | |
| | Result: | NOT DIFFERENT | Result: | SIMILAR |

*not used for calculations only 1 point above 85% used

TABLE 26
F1 and F2 calculation using normalized data for 12.5 mg fondaparinux tablet

| F1 calculation | | | | | | |
|---|---|---|---|---|---|---|
| | C10 | | | | | |
| time | (reference) | Fondaparinux | | | | |
| [t] | pre-change | post-change | | | F2 calculation | |
| min | [Rt] | [T] | R − T | ABS [R − T] | | (R − T)² |
| 5 | 0.0 | 0.0 | 0.0 | 0 | | 0.0 |
| 10 | 4.0 | 0.0 | 4.0 | 4.04040404 | | 16.32 |
| 20 | 29.9 | 29.0 | 0.9 | 0.9341276 | | 0.87 |
| 25 | 50.0 | 47.2 | 2.8 | 2.80151947 | | 7.85 |
| 30 | 66.7 | 64.0 | 2.7 | 2.65906933 | | 7.07 |
| 35 | 80.3 | 78.3 | 2.1 | 2.05041872 | n (timepoints) = 7 | 4.20 |
| 45 | 96.1 | 94.6 | 1.5 | 1.47371147 | | 2.17 |
| *60 | 100.0 | 100.0 | 0.0 | 0 | SUM(R − T)² | 38.49 |
| sum | 327.0 | | | | (1/n) | 0.142857143 |
| | | | Sum ABS [R − T] | 14.0 | (1/n) * SUM(R − T)² | 5.498951802 |
| | | | f1 Factor | 4.26927962 | 1 + [1/n) * SUM(R − T)² | 6.498951802 |

| n (timepoints) = 7.0 | | | SQRT | 2.54930418 |
|---|---|---|---|---|
| | | | (1/SQRT) * 100 | 39.22639 |
| | | | F2 FACTOR | 79.6789171 |
| USP Guidance: Not different if (f1) in range from 0-15 | | | USP Guidance: Similar if (f2) in range | |
| | | | of 50-100 | |
| | Result: | NOT DIFFERENT | Result: | SIMILAR |

*not used for calculations only 1 point above 85% used

The results of the dissolution study for the tablet of Test Item 2 (20 mg fondaparinux) are shown in Tables 27 (fondaparinux) and 28 (sodium caprate).

TABLE 27
Dissolution of fondaparinux from 20 mg fondaparinux tablet
% Dissolution Results

| Minutes | Acid | 5 | 10 | 20 | 25 | 30 | 35 | 45 | 60 |
|---|---|---|---|---|---|---|---|---|---|
| V1 | ND | ND | ND | 23.5 | 46.0 | 65.0 | 81.0 | 94.0 | 95.9 |
| V2 | ND | ND | ND | 39.1 | 60.6 | 82.6 | 89.2 | 101.4 | 103.8 |
| V3 | ND | ND | ND | 28.7 | 54.0 | 72.6 | 83.9 | 96.8 | 100.6 |
| V4 | ND | ND | ND | 44.0 | 68.0 | 86.5 | 91.5 | 100.7 | 101.0 |
| V5 | ND | ND | ND | 40.6 | 66.3 | 86.4 | 93.7 | 97.6 | 101.5 |
| V6 | ND | ND | ND | 31.7 | 67.7 | 73.6 | 88.1 | 101.4 | 103.5 |
| Mean: | N/A | N/A | N/A | 34.6 | 60.5 | 77.8 | 87.9 | 98.6 | 101.1 |
| % RSD: | N/A | N/A | N/A | 22.7 | 14.7 | 11.2 | 5.4 | 3.1 | 2.8 |

ND = Not Detected
N/A = Not Applicable

TABLE 28
Dissolution of sodium caprate from 20 mg fondaparinux tablet
% Dissolution Results

| Minutes | Acid | 5 | 10 | 20 | 25 | 30 | 35 | 45 | 60 |
|---|---|---|---|---|---|---|---|---|---|
| V1 | ND | ND | 1.9 | 27.1 | 46.7 | 64.10 | 77.1 | 93.7 | 99.5 |
| V2 | ND | ND | 3.7 | 37.3 | 54.0 | 69.4 | 80.8 | 93.9 | 97.9 |
| V3 | ND | ND | 4.2 | 33.0 | 50.1 | 65.6 | 78.0 | 94.1 | 99.0 |
| V4 | ND | ND | 4.0 | 43.0 | 62.1 | 77.6 | 87.9 | 97.3 | 98.9 |
| V5 | ND | ND | 4.3 | 41.3 | 61.6 | 79.7 | 89.0 | 96.7 | 99.5 |
| V6 | ND | ND | 2.3 | 32.0 | 50.6 | 67.4 | 80.3 | 97.0 | 99.3 |
| Mean: | N/A | N/A | 3.4 | 35.6 | 54.2 | 70.6 | 82.2 | 95.4 | 99.0 |
| % RSD: | N/A | N/A | 30.3 | 17.0 | 11.8 | 9.2 | 6.1 | 1.8 | 0.6 |

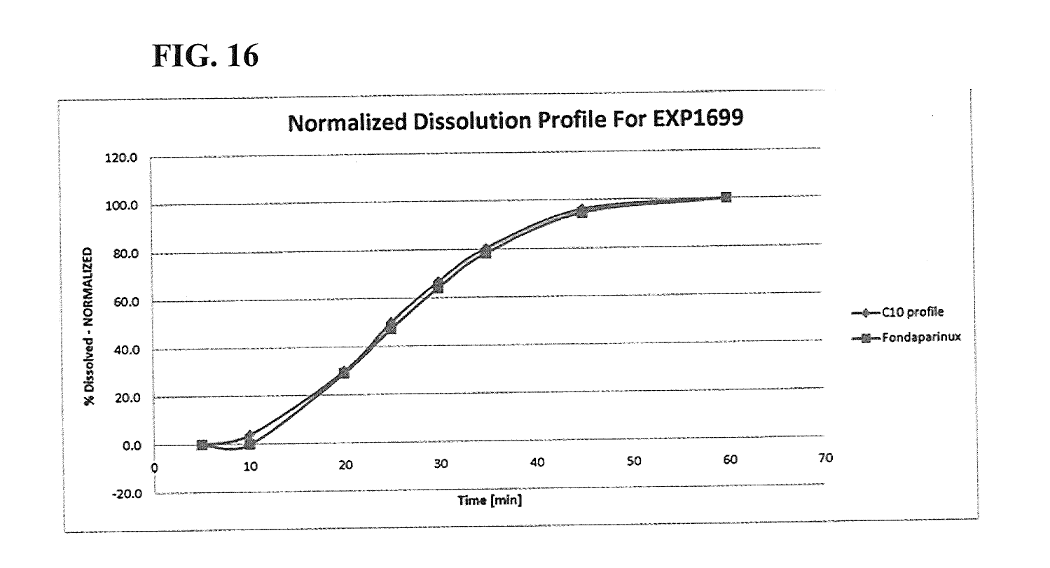

The dissolution data were normalized and the normalized results are shown in Table 29 and plotted in FIG. 16.

TABLE 29
Normalized dissolution data for 20 mg fondaparinux tablet

| Time | % Disso C10 | % Disso Fondaparinux |
|---|---|---|
| 5 | 0.0 | 0.0 |
| 10 | 3.4 | 0.0 |
| 20 | 36.0 | 34.2 |
| 25 | 54.7 | 59.8 |
| 30 | 71.3 | 77.0 |
| 35 | 83.0 | 86.9 |
| 45 | 96.4 | 97.5 |
| 60 | 100.0 | 100.0 |

The dissolution data was used to calculate the difference (f1) and similarity (f2) factors. The results are shown in Tables 30 (non-normalized data) and 31 (normalized data). Using both the normalized and non-normalized data, the dissolution rate of sodium caprate and fondaparinux were calculated to be highly similar.

TABLE 30
F1 and F2 calculation using non-normalized data for 20 mg fondaparinux
tablet

| F1 calculation | | | | | | |
|---|---|---|---|---|---|---|
| | C10 | | | | | |
| time | (reference) | Fondaparinux | | | | |
| [t] | pre-change | post-change | | | F2 calculation | |
| min | [Rt] | [T] | R − T | ABS [R − T] | | (R − T)² |
| 5 | 0.0 | 0.0 | 0.0 | 0 | | 0.00 |
| 10 | 3.4 | 0.0 | 3.4 | 3.4 | | 11.56 |
| 20 | 35.6 | 34.6 | 1.0 | 1 | | 1.00 |
| 25 | 54.2 | 60.5 | −6.3 | 6.3 | | 39.69 |
| 30 | 70.6 | 77.8 | −7.2 | 7.2 | | 51.84 |
| 35 | 82.2 | 87.9 | −5.7 | 5.7 | | 32.49 |
| 45 | 95.4 | 98.6 | −3.2 | 3.2 | n (timepoints) = 7 | 10.24 |
| *60 | 99.0 | 101.1 | | | SUM(R − T)² | 146.82 |
| sum* (5-45 min) | 341.4 | | | | (1/n) | 0.142857143 |
| | | | Sum ABS [R − T] | 26.8 | (1/n) * SUM(R − T)² | 20.97428571 |
| | | | f1 Factor | 7.85002929 | 1 + [1/n) * SUM(R − T)² | 21.97428571 |

| | | |
|---|---|---|
| n (timepoints) = 7.0 | SQRT | 4.687673806 |
| | (1/SQRT) * 100 | 21.33254235 |

| | | |
|---|---|---|
| USP Guidance: Not different if (f1) in range from 0-15 | F2 FACTOR | 66.45213082 |

| | USP Guidance: Similar if (f2) in range | |
|---|---|---|
| | of 50-100 | |
| Result: NOT DIFFERENT | Result: | SIMILAR |

*not used for calculations only 1 point above 85% used

TABLE 31
F1 and F2 calculation using normalized data for 20 mg fondaparinux tablet

| | C10 | | | | | |
|---|---|---|---|---|---|---|
| time | (reference) | Fondaparinux | | | | |
| [t] | pre-change | post-change | | | | |
| min | [Rt] | [T] | R − T | ABS [R − T] | | (R − T)² |
| 5 | 0.0 | 0.0 | 0.0 | 0 | | 0.0 |
| 10 | 3.4 | 0.0 | 3.4 | 3.434343434 | | 11.79 |
| 20 | 36.0 | 34.2 | 1.7 | 1.736054911 | | 3.01 |
| 25 | 54.7 | 59.8 | −5.1 | 5.094266103 | | 25.95 |
| 30 | 71.3 | 77.0 | −5.6 | 5.640380062 | | 31.81 |
| 35 | 83.0 | 86.9 | −3.9 | 3.913317148 | | 15.31 |
| 45 | 96.4 | 97.5 | −1.2 | 1.163564428 | n (timepoints) = 7 | 1.35 |
| *60 | 100.0 | 100.0 | 0.0 | 0 | SUM(R − T)² | 89.24 |
| sum | 344.8 | | | | (1/n) | 0.142857143 |
| | | | Sum ABS [R − T] | 21.0 | (1/n) * SUM(R − T)² | 12.74885273 |
| | | | f1 Factor | 6.084389814 | 1 + [1/n) * SUM(R − T)² | 13.74885273 |

| | | |
|---|---|---|
| n (timepoints) = 7.0 | SQRT | 3.707944543 |
| | (1/SQRT) * 100 | 26.96911964 |

| | | |
|---|---|---|
| USP Guidance: Not different if (f1) in range from 0-15 | F2 FACTOR | 71.5433385 |

| | | USP Guidance: Similar if (f2) in | |
|---|---|---|---|
| | | range of 50-100 | |
| Result: | NOT DIFFERENT | Result: | SIMILAR |

*not used for calculations only 1 point above 85%

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A pharmaceutical composition for oral administration, comprising a therapeutically effective amount of a selective factor Xa inhibitor or a pharmaceutically acceptable salt thereof and an enhancer, wherein the enhancer is a medium chain fatty acid or a salt, ester, ether, or derivative of a medium chain fatty acid and has a carbon chain length of from 4 to 20 carbon atoms.

2. The pharmaceutical composition of claim 1, wherein said selective factor Xa inhibitor is an oligosaccharide.

3. The pharmaceutical composition of claim 1, wherein said selective factor Xa inhibitor is a pentasaccharide.

4. The pharmaceutical composition of claim 1, wherein said selective factor Xa inhibitor is fondaparinux or a pharmaceutically acceptable salt thereof.

5. (canceled)

6. The pharmaceutical composition of claim 1, wherein the enhancer is a salt of a medium chain fatty acid and is solid at room temperature.

7. The pharmaceutical composition of claim 1, wherein the carbon chain length is from 8 to 14 carbon atoms.

8. The pharmaceutical composition of claim 1, wherein the enhancer is a sodium salt of a medium chain fatty acid.

9. The pharmaceutical composition of claim 1, wherein the enhancer is selected from the group consisting of sodium caprylate, sodium caprate and sodium laurate.

10. (canceled)

11. A solid oral dosage form comprising the pharmaceutical composition of claim 1.

12. The solid oral dosage form of claim 11, wherein the dosage form is a tablet, a capsule or a multiparticulate dosage form.

13. The solid oral dosage form of claim 11, wherein the dosage form is a controlled release dosage form.

14. The solid oral dosage form of claim 12, wherein the dosage form further comprises a rate controlling polymer material.

15-16. (canceled)

17. The solid oral dosage form of claim 14, wherein the selective factor Xa inhibitor and enhancer and at least one auxiliary excipient are compressed into tablet form prior to coating with a rate controlling polymer or delayed release polymer.

18-42. (canceled)

43. The pharmaceutical composition of claim 1, wherein said pharmaceutical composition provides a bioavailability of the selective factor Xa inhibitor or a pharmaceutically acceptable salt thereof of at least about 10% when orally administered to a human subject or a dog.

44-46. (canceled)

47. The pharmaceutical composition of claim 1, wherein the bioavailability of the selective factor Xa inhibitor or a pharmaceutically acceptable salt thereof has a coefficient of variation of no more than about 60% when orally administered to a human subject or a dog.

48-50. (canceled)

51. A pharmaceutical composition comprising a selective factor Xa inhibitor or a pharmaceutically acceptable salt thereof, wherein said pharmaceutical composition provides a bioavailability of the selective factor Xa inhibitor or a pharmaceutically acceptable salt thereof of at least about 10% when orally administered to a human subject or a dog.

52-54. (canceled)

55. A solid oral dosage form comprising the pharmaceutical composition of claim 51.

56. The pharmaceutical composition of claim 1, wherein the pharmaceutical composition provides a substantially similar release rate of the selective factor Xa inhibitor and the enhancer after the pharmaceutical composition enters the intestine of a subject, wherein the dissolution is measured in 900 mL pH 6.8 phosphate buffer at 37° C. with a USP Paddle Apparatus at 50 rpm.

57-58. (canceled)

59. The pharmaceutical composition or solid oral dosage form of claim 56 wherein f1 for the dissolution profile of the enhancer and the selective factor Xa inhibitor is less than about 15.

60. The pharmaceutical composition or solid oral dosage form of claim 56, wherein f2 for the dissolution profile of the enhancer and the selective factor Xa inhibitor is in a range of about 50 to about 100.

61-65. (canceled)

66. A method of treating or preventing a medical condition, comprising administering to a subject in need of such treatment or prevention the pharmaceutical composition of claim 1.

67. The method of claim 66, wherein said medical condition is a thromboembolic condition.

68. A method for obtaining a reproducible bioavailability of selective factor Xa inhibitor in a subject after oral administration, comprising orally administering to said subject the pharmaceutical composition of claim 1.

69-71. (canceled)
